(19) United States
(12) Patent Application Publication
(10) Pub. No.: US 2022/0056425 A1
(43) Pub. Date: Feb. 24, 2022

(54) RNA POLYMERASE FOR SYNTHESIS OF MODIFIED RNA

(71) Applicant: GUARDIAN THERAPEUTICS, LLC, Lexington, MA (US)

(72) Inventors: Kristin THOMPSON, Arlington, MA (US); Yuxun WANG, Shrewsbury, MA (US); Shuhao ZHU, Concord, MA (US)

(21) Appl. No.: 17/275,740

(22) PCT Filed: Sep. 12, 2019
(86) PCT No.: PCT/US2019/050777

§ 371 Date: Mar. 12, 2021

Related U.S. Application Data
(60) Provisional application No. 62/730,226, filed on Sep. 12, 2018.

(51) Int. Cl. C12N 9/12; C12N 15/115

(52) U.S. Cl. CPC C12N 9/1247; C12N 15/115; C12N 2330/50; C12N 2310/322; C12N 2310/16

(57) ABSTRACT

The present disclosure provides T7 RNA polymerase variants with enhanced transcriptional activity, and methods of using such variants to produce modified oligonucleotides, such as 2′-modified oligonucleotides. These polymerase variants and methods thereof improve the transcription yield of modified oligonucleotides.

### REFERENCES TO RELATED APPLICATION

The present application claims priority to U.S. Provisional Application No. 62/730,226 filed Sep. 12, 2018, entitled “RNA polymerase for synthesis of modified RNA”; the contents of which are incorporated herein by reference in their entirety.

### SEQUENCE LISTING

The present application is being filed along with a Sequence Listing in electronic format. The Sequence Listing file, entitled SEQLST_2125-1000PCT.txt, was created on Sep. 12, 2019, and is 41,166 bytes in size. The information in electronic format of the Sequence Listing is incorporated herein by reference in its entirety.

### FIELD OF THE DISCLOSURE

The present disclosure relates to materials, reagents, methods, and kits for the preparation of polynucleotides. Specifically, the present disclosure provides RNA polymerase variants and related methods for the preparation of modified polynucleotides, for example, 2′-modified polynucleotides. The present disclosure also relates to the 2′modified polynucleotides produced according to the methods of the disclosure.

### BACKGROUND OF THE DISCLOSURE

Modified nucleic acids, especially modifications of 2′ position of the ribose backbone, have novel properties that their natural counterparts do not possess. For example, 2′-O-methylation, where the ribose 2′ hydroxyl moiety is substituted by an O-methyl (“2-OMe”) group, is commonly used as a solution for nuclease stability issues or the duplex stability of DNA molecules. 2′-O-methyl RNA has enhanced stability against general base hydrolysis and nucleases. 2′-O-methyl modification can increase the melting temperature (Tm) of duplexes by 1-4° C. per addition. Moreover, small interfering RNA (siRNA) with 2′-fluoro and 2′-O-methyl RNA has also shown to be less immunogenic, more stable in serum and more target-specific (see, e.g., Kenski et al., SiRNA-optimized modifications for Enhanced in vivo activity. Mol Ther Nucleic Acids. 2012; 1: e5). However, 2′-modified nucleotide insertions are not recognized by many polymerases, thereby limiting their utility.

Nucleic acid aptamers are a new class of high-affinity ligands. Aptamers are stable, inexpensive and non-toxic. Aptamers containing modified nucleotides (e.g., 2′-modified nucleotides) are used to increase resistance to enzymatic, chemical, thermal, and physical degradation of aptamers. Aptamers may be generated using a process known as “Systematic Evolution of Ligands by Exponential Enrichment” (“SELEX™”). The SELEX™ process is a method for the in vitro evolution of nucleic acid molecules with highly specific binding to target molecules and is described in, e.g., U.S. patent application Ser. No. 07/536,428, U.S. Pat. Nos. 5,475,096 and 5,270,163. Modified nucleotides (e.g., 2′-modified nucleotides) can be incorporated into aptamer sequences during SELEX™ method as described in U.S. Pat. No. 7,022,144, and U.S. patent application Ser. No. 10/873,856. While incorporation of modified nucleotides during SELEX™ process is often times preferable to post SELEX™ modification due to potential loss of binding affinity and activity that can occur post-SELEX™ selection, the incorporation of modified nucleotides, e.g. 2′-O-methyl nucleotides, during the SELEX™ process has been historically difficult because of low transcription yields with regular enzymatic agents.

As such, there is a need for developing new polymerases with improved activity and methods for increasing the transcription yield of modified oligonucleotides. The present disclosure screened RNA polymerase variants and identified T7 RNA polymerase variants with increased ability to incorporated modified nucleotides, particularly 2′-modified nucleotides.

### SUMMARY OF THE DISCLOSURE

The present disclosure provides materials, reagents, methods and kits for the synthesis of modified oligonucleotides such as aptamers.

One aspect of the present disclosure relates to T7 RNA polymerase variants. In some embodiments, provided herein is a T7 RNA polymerase variant having one or more mutations relative to the wild-type polypeptide sequence set forth in SEQ ID NO.: 1, wherein the one or more mutations include: a) at least one amino acid substitution chosen from K378R, Y639L, and H784A; and b) at least one amino acid substitution chosen from S430P, N433T, S633P, F849I, F880Y and P266L.

In some embodiments, the T7 RNA polymerase variant has a) one of the following two sets of amino acid substitutions: i) Y639L, and H784A; or ii) K378R, Y639L and H784A; and b) one of the following two sets of amino acid substitutions: i) S430P, N433T, S633P, F849I and F880Y; or ii) S430P, N433T, S633P, F849I, F880Y and P266L.

In some embodiments, the T7 RNA polymerase variant has a) K378R, Y639L and H784A; and b) S430P, N433T, S633P, F849I and F880Y.

In some embodiments, the T7 RNA polymerase variant has a) K378R, Y639L and H784A; and b) S430P, N433T, S633P, F849I, F880Y and P266L.

In some embodiments, T7 RNA polymerase variants provided herein further comprise a protein tag. In some embodiments, the protein tag is attached to the N-terminus of the polymerase variant. In some embodiments, the protein tag is attached to the C-terminus of the polymerase variant. In some embodiments, the protein tag is a polyhistidine-tag. In one embodiment, the protein tag is a hexa histidine-tag.

In one embodiment, the T7 RNA polymerase variant has a polypeptide sequence set forth in SEQ ID NO.: 2, or a functional fragment thereof.

In one embodiment, the T7 RNA polymerase variant has a polypeptide sequence set forth in SEQ ID NO.: 5, or a functional fragment thereof.

In some embodiments, T7 RNA polymerase variants provided herein have enhanced ability to incorporate a 2′ modified nucleotide compared to the wild-type T7 RNA polymerase. In some embodiments, the 2′ modified nucleotide may be selected from 2′-O-methyl ATP, 2′-O-methyl CTP, 2′-O-methyl UTP, 2′-O-methyl GTP, 2′-fluoro ATP, 2′-fluoro CTP, 2′-fluoro UTP, 2′-fluoro GTP, 2′-amino ATP, 2′-amino CTP, 2′-amino UTP, and 2′-amino GTP. In one embodiment, the 2′ modified nucleotide may be 2′-O-methyl ATP, 2′-O-methyl CTP, 2′-O-methyl UTP, or 2′-O-methyl GTP.

In some embodiments, T7 RNA polymerase variants provided herein do not have a bias in incorporating 2′-O-methyl ATP, 2′-O-methyl CTP, 2′-O-methyl UTP, or 2′-O-methyl GTP.

In some embodiments, T7 RNA polymerase variants provided herein have enhanced thermostability compared to the wild-type T7 RNA polymerase.

Also provided herein is a nucleic acid molecule encoding a T7 RNA polymerase variant of the present disclosure. The sequence of the nucleic acid molecule may be codon optimized for expression in a protein expression system. In one embodiment, the sequence of the nucleic acid molecule may be codon optimized for expression in an E. coli expression system.

In one embodiment, the nucleic acid molecule encoding a T7 RNA polymerase variant has a sequence corresponding to SEQ ID NO.: 3.

In one embodiment, the nucleic acid molecule encoding a T7 RNA polymerase variant has a sequence corresponding to SEQ ID NO.: 6.

Also provided herein is an expression vector comprising the nucleic acid molecule encoding a T7 RNA polymerase variant of the present disclosure. In some embodiments, the expression vector may comprise a nucleic acid sequence set forth in SEQ ID NO.: 3. In other embodiments, the expression vector may comprise a nucleic acid sequence set forth in SEQ ID NO.: 6.

Also provided herein is a cell comprising the expression vector capable of expressing the T7 RNA polymerase variant of the present disclosure. In some embodiments, the cell comprises a nucleic acid molecule having a sequence corresponding to SEQ ID NO.: 3 that encodes a T7 RNA polymerase variant. In other embodiments, the cell comprises a nucleic acid molecule having a sequence corresponding to SEQ ID NO: 6 that encodes a T7 RNA polymerase variant.

In some embodiments, a production system comprising the cell expressing the T7 RNA polymerase variant is further provided.

Also provided herein is a kit comprising a T7 RNA polymerase variant of the present disclosure for use with a method of the present disclosure.

Further provided herein is a method of synthesizing a 2′ modified polynucleotide, including contacting a template nucleic acid with a T7 RNA polymerase variant of the present disclosure in the presence of 2′ modified nucleoside triphosphates under conditions that allow synthesis of the 2′-modified polynucleotide by the polymerase activity of the T7 RNA polymerase variant.

In some embodiments, the 2′ modified nucleoside triphosphates are one or more nucleoside triphosphates selected from 2′-O-methyl ATP, 2′-O-methyl CTP, 2′-O-methyl UTP, 2′-O-methyl GTP, 2′-fluoro ATP, 2′-fluoro CTP, 2′-fluoro UTP, 2′-fluoro GTP, 2′-amino ATP, 2′-amino CTP, 2′-amino UTP, 2′-amino GTP, and/or combinations thereof.

In some embodiments, the 2′ modified nucleoside triphosphates are one or more nucleoside triphosphates chosen from 2′-O-methyl ATP, 2′-O-methyl CTP, 2′-O-methyl UTP, 2′-O-methyl GTP, and/or combinations thereof.

Further provided herein is a polynucleotide comprising at least one 2′ modified nucleotide residue.

In some embodiments, the 2′ modified polynucleotide is a nucleic acid aptamer, a ribozyme, an siRNA, a pre-miRNA, a miRNA, or an antisense RNA. In one embodiment, the 2′ modified polynucleotide is a nucleic acid aptamer.

In some embodiments, the 2′ modified polynucleotide is a resistant to nucleases and/or base hydrolysis.

Further provided herein is use of a T7 RNA polymerase variant of the present disclosure for the synthesis of a 2′ modified polynucleotide. In some embodiments, a T7 RNA polymerase variants of the present disclosure is used in the SELEX™ process for the synthesis of a 2′ modified polynucleotide.

Further provided herein is a nucleic acid aptamer, comprising one or more 2′ modified nucleotide residues, synthesized according to a method described herein. Said modified nucleic acid aptamer can bind to a target of interest specifically.

### BRIEF DESCRIPTION OF THE DRAWINGS

The foregoing and other objects, features and advantages will be apparent from the following description of particular embodiments of the disclosure, as illustrated in the accompanying drawings. The drawings are not necessarily to scale; emphasis instead being placed upon illustrating the principles of various embodiments of the disclosure.

FIG. 1 shows polyacrylamide gel electrophoresis (SDS-PAGE) analysis of the purified T7 RNA polymerase variants.

FIG. 2A and FIG. 2B show results from two sets of in vitro transcription reactions.

FIG. 3 is a diagram demonstrating the plate-based binding assay to validate the binding of selected aptamers with their target proteins.

FIG. 4 shows Cholera Toxin binding characteristics of modified aptamer candidates.

FIG. 5 shows the functional activity of Cholera Toxin (subunit B or holotoxin) in competition assay with modified aptamer candidates.

FIG. 6 shows the binding characteristics of modified aptamer candidates (3 clones) to recombinant 4-1BB-Fc-His protein.

FIG. 7 illustrates the binding assay of two positive candidates to recombinant KRAS protein.

### DETAILED DESCRIPTION OF THE DISCLOSURE

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

### I. Introduction

Several variants of T7 RNA polymerase have been developed to promote the incorporation of 2′modified nucleotides (e.g., 2′-O-methyl nucleotides, 2′-deoxy-nucleotides or 2′-fluoro-nucleotides) into RNA transcripts at a rate higher than that of wild-type T7 RNA polymerase. Such variants have been described in Sousa and Padilla, EMBO J. 1995; 14(18):4609-4621; Padilla and Sousa, Nucleic Acids Res., 2002, 30(24): e138; Burmeister et al., Chem Biol. 2005; 12(1):25-33; Chelliserrykattil and Ellington, Nature Biotech, 2004; 22(9):1155-1160; Guillerez et al., Proc Natl Acad Sci USA. 2005; 102(17): 5958-5963; U.S. Pat. Nos. 8,105,813; 7,507,567; Meyer et al., Nucleic Acids Res. 2015; 43(15):7480-7488; U.S. Pat. Nos. 8,105,813; 9,193,959; and 9,988,612; the contents of each of which are herein incorporated by reference in their entirety.

While existing T7 RNA polymerase variants may be capable of incorporating 2′ modified nucleotides, most of them suffer from low activity. It has been described that activity of the T7 RNA polymerase variants is often sacrificed for increased substrate specificity conferred by introduced mutations, leading to low transcript yields (Padilla and Sousa, Nucleic Acids Res. 1999; 27(6):1561-1563). The mutations that confer new activity in the polymerase may also destabilize the protein, rendering it less active overall. As such, there is a need for developing new enzymes with improved activity and methods for increasing the transcription yield of modified oligonucleotides.

### II. Compositions and Methods of the Disclosure

The present disclosure provides materials, reagents, methods, and kits for synthesis of modified oligonucleotides. Particularly, provided herein are T7 RNA polymerase variants that are capable of producing high yields of modified oligonucleotides (e.g., 2′-O-methylated oligonucleotides). Also provided herein are methods and conditions for using the T7 RNA polymerase variants for the transcription of oligonucleotides. Further provided herein are modified polynucleotides (e.g., 2′-O-methylated oligonucleotides) produced by the materials, enzymatic reagents and methods described herein.

The present disclosure provides a group of novel T7 RNA polymerases with increased ability for insertion of 2′-O-modified nucleotides.

### Polymerase Variants

The term “polypeptide” or “protein”, as used herein, refers to a polymer composed of a plurality of amino acid monomers joined by peptide bonds. In some embodiments, a polypeptide or protein according to the disclosure is a T7 RNA polymerase (wild-type, mutant or variant). A “peptide bond” is a covalent bond between a first amino acid and a second amino acid in which the α-amino group of the first amino acid is bonded to the α-carboxyl group of the second amino acid.

The term “variant” or “mutant”, as used interchangeably herein, refers to derivatives of a protein or polypeptide that comprise modifications of the amino acid sequence, for example, by substitution, deletion, insertion or chemical modification.

“T7 RNA polymerase” is the DNA-directed RNA polymerase of bacteriophage T7 (enterobacteria phage T7). The amino acid sequence of the wild-type T7 RNA polymerase sequence is provided below:

(SEQ ID NO.: 1)
MNTINIAKNDFSDIELAAIPFNTLADHYGERLAREQLALEHESYEMGE
ARFRKMFERQLKAGEVADNAAAKPLITTLLPKMIARINDWFEEVKAKR
GKRPTAFQFLQEIKPEAVAYITIKTTLACLTSADNTTVQAVASAIGRA
IEDEARFGRIRDLEAKHFKKNVEEQLNKRVGHVYKKAFMQVVEADMLS
KGLLGGEAWSSWHKEDSIHVGVRCIEMLIESTGMVSLHRQNAGVVGQD
SETIELAPEYAEAIATRAGALAGISPMFQPCVVPPKPWTGITGGGYWA
NGRRPLALVRTHSKKALMRYEDVYMPEVYKAINIAQNTAWKINKKVLA
VANVITKWKHCPVEDIPAIEREELPMKPEDIDMNPEALTAWKRAAAAV
YRKDKARKSRRISLEFMLEQANKFANHKAIWFPYNMDWRGRVYAVSMF
NPQGNDMTKGLLTLAKGKPIGKEGYYWLKIHGANCAGVDKVPFPERIK
FIEENHENEVIACAKSPLENTWWAEQDSPFCFLAFCFEYAGVQHHGLS
YNCSLPLAFDGSCSGIQHFSAMLRDEVGGRAVNLLPSETVQDIYGIVA
KKVNEILQADAINGTDNEVVTVTDENTGEISEKVKLGTKALAGQWLAY
GVTRSVTKRSVMTLAYGSKEFGFRQQVLEDTIQPAIDSGKGLMFTQPN
QAAGYMAKLIWESVSVTVVAAVEAMNWLKSAAKLLAAEVKDKKTGEIL
RKRCAVHWVTPDGFPVWQEYKKPIQTRLNLMFLGQFRLQPTINTNKDS
EIDAHKQESGIAPNFVHSQDGSHLRKTVVWAHEKYGIESFALIHDSFG
TIPADAANLFKAVRETMVDTYESCDVLADFYDQFADQLHESQLDKMPA
LPAKGNLNLRDILESDFAFA

In some embodiments, the present disclosure provides T7 RNA polymerase variants. The T7 RNA polymerases variants described herein are capable of incorporating modified nucleotide substrates, e.g., 2′-modified nucleotides. 2′-modified nucleotides may include, but are not limited to, 2′ deoxy nucleotides, 2′ amino (2′-NH₂)-modified nucleotides (e.g., 2′-amino ATP, 2′-amino CTP, 2′-amino UTP, and 2′-amino GTP), 2′ fluoro (2′-F)-modified nucleotides (e.g., 2′-fluoro ATP, 2′-fluoro CTP, 2′-fluoro UTP, and 2′-fluoro GTP), 2′-O-Methyl (2′-OMe)-modified nucleotides, 2′-azido (2′-N3)-modified nucleotides, and 2′-O-(2-Methoxyethyl) (2′-O-MOE)-modified nucleotides. In some embodiments, the 2′-modified nucleotides are 2′-O-Methyl (2′-OMe)-modified nucleotides, including but not limited to 2′-O-methyl ATP, 2′-O-methyl CTP, 2′-O-methyl UTP, and 2′-O-methyl GTP.

In some embodiments, the nucleotides may contain a natural or unmodified nucleobase (often referred to in the art simply as “base”). As used herein, “unmodified” or “natural” nucleobases include the purine bases adenine (A) and guanine (G), and the pyrimidine bases thymine (T), cytosine (C) and uracil (U). In other embodiments, the nucleotides may contain a modified nucleobase. Modified nucleobases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30: 613, and those disclosed by Sanghvi, Y S., Chapter 15, dsRNA Research and Applications, pages 289-302, Crooke, S. T. and Lebleu, B., Ed., CRC Press, 1993; the contents of each of which are incorporated herein by reference in their entirety. In some embodiments, the nucleotides may contain a combination of natural bases and modified bases.

In some embodiments, the T7 RNA polymerase variant may comprise one or more mutations that allow the polymerase to accept a 2′-modified nucleotide. In some embodiments, such mutations may include amino acid substitutions such as, but not limited to, K378R, Y639L, Y639F, and H784A. In some embodiments, the T7 RNA polymerase variant may comprise a low-abortive mutation such as P266L. As non-limiting examples, the T7 RNA polymerase variant may comprise any one set of the following mutations: Y639F; Y639F and K378R; Y639F and H784A; Y639F, H784A and K378R; Y639L; Y639L and K378R; Y639L and H784A; Y639L, H784A and K378R; P266L, Y639L and H784A; or P266L, Y639L, H784A and K378R. Other T7 RNA polymerase mutations, particularly those that facilitate a high tolerance for bulky 2′-substituents (e.g., A255T, G542V, E593G, Y639V, V685A, H772R, H784G, H784S), may also be included in the T7 RNA polymerase variants of the present disclosure. In one embodiment, the T7 RNA polymerase variant comprises Y639L, H784A and K378R. In one embodiment, the T7 RNA polymerase variant comprises P266L, Y639L, H784A and K378R.

In some embodiments, the T7 RNA polymerase variant may comprise one or more mutations that enhance the thermostability of the polymerase. In some embodiments, a T7 RNA polymerase variant includes V426L, A702V and V795I mutations. In some embodiments, a T7 RNA polymerase variant includes S430P, F849I, S633I and F880Y mutations. In some embodiments, a T7 RNA polymerase variant includes F880Y, S430P, F849I, S633I, C510R and S767G mutations. In some embodiments, a T7 RNA polymerase variant includes Y639V, H784G, E593G and V685A mutations. In some embodiments, a T7 RNA polymerase variant includes S430P, N433T, S633P, F849I and F880Y mutations. Other variants and recombinant thermostable polymerases are encompassed by the present disclosure.

In some embodiments, the T7 RNA polymerase variant comprises one or more mutations selected from the group consisting of Y639L, H784A, K378R, S430P, N433T, S633P, F849I and F880Y.

In some embodiments, the T7 RNA polymerase variant comprises one or more mutations selected from the group consisting of Y639L, H784A, K378R, S430P, N433T, S633P, F849I, F880Y and P266L.

In one embodiment, the T7 RNA polymerase variant comprises Y639L, H784A, K378R, S430P, N433T, S633P, F849I and F880Y. This variant is herein termed “LAR-M5.”

In one embodiment, the T7 RNA polymerase variant comprises Y639L, H784A, K378R, S430P, N433T, S633P, F849I, F880Y and P266L. This variant is herein termed “LAR-M6.”

The amino acid sequence corresponding to the LAR-M5 variant is provided below:

(SEQ ID NO.: 2)
MNTINIAKNDFSDIELAAIPFNTLADHYGERLAREQLALEHESYEMGE
ARFRKMFERQLKAGEVADNAAAKPLITTLLPKMIARINDWFEEVKAKR
GKRPTAFQFLQEIKPEAVAYITIKTTLACLTSADNTTVQAVASAIGRA
IEDEARFGRIRDLEAKHFKKNVEEQLNKRVGHVYKKAFMQVVEADMLS
KGLLGGEAWSSWHKEDSIHVGVRCIEMLIESTGMVSLHRQNAGVVGQD
SETIELAPEYAEAIATRAGALAGISPMFQPCVVPPKPWTGITGGGYWA
NGRRPLALVRTHSKKALMRYEDVYMPEVYKAINIAQNTAWKINKKVLA
VANVITKWKHCPVEDIPAIEREELPMKPEDIDMNPEALTAWRRAAAAV
YRKDKARKSRRISLEFMLEQANKFANHKAIWFPYNMDWRGRVYAVPMF
TPQGNDMTKGLLTLAKGKPIGKEGYYWLKIHGANCAGVDKVPFPERIK
FIEENHENEVIACAKSPLENTWWAEQDSPFCFLAFCFEYAGVQHHGLS
YNCSLPLAFDGSCSGIQHFSAMLRDEVGGRAVNLLPSETVQDIYGIVA
KKVNEILQADAINGTDNEVVTVTDENTGEISEKVKLGTKALAGQWLAY
GVTRSVTKRPVMTLALGSKEFGFRQQVLEDTIQPAIDSGKGLMFTQPN
QAAGYMAKLIWESVSVTVVAAVEAMNWLKSAAKLLAAEVKDKKTGEIL
RKRCAVHWVTPDGFPVWQEYKKPIQTRLNLMFLGQFRLQPTINTNKDS
EIDAHKQESGIAPNFVASQDGSHLRKTVVWAHEKYGIESFALIHDSFG
TIPADAANLFKAVRETMVDTYESCDVLADFYDQIADQLHESQLDKMPA
LPAKGNLNLRDILESDYAFA.

In some embodiments, the nucleic acid sequence encoding a T7 RNA polymerase variant is codon-optimized for a protein expression system. For example, the expression system may be a bacterial expression system, such as E. coli expression system.

As a non-limiting example, the nucleic acid sequence encoding the LAR-M5 variant may be the nucleic acid sequence provided below:

(SEQ ID NO.: 3)
ATGAACACAATAAACATAGCTAAGAACGACTTTAGCGACATCGAACTG
GCGGCGATTCCGTTTAACACCCTGGCGGACCACTATGGCGAGCGTCTG
GCGCGTGAACAGCTGGCGCTGGAGCACGAAAGCTATGAGATGGGTGAA
GCGCGTTTCCGTAAGATGTTTGAGCGTCAACTGAAAGCGGGCGAAGTT
GCGGACAACGCGGCGGCGAAGCCGCTGATTACCACCCTGCTGCCGAAA
ATGATCGCGCGTATTAACGATTGGTTCGAGGAAGTTAAGGCGAAACGT
GGCAAGCGTCCGACCGCGTTCCAGTTTCTGCAAGAGATCAAGCCGGAA
GCGGTGGCGTACATCACCATTAAAACCACCCTGGCGTGCCTGACCAGC
GCGGACAACACCACCGTTCAAGCGGTTGCGAGCGCGATTGGTCGTGCG
ATTGAGGACGAAGCGCGTTTTGGCCGTATTCGTGATCTGGAGGCGAAG
CACTTCAAGAAAAACGTTGAGGAACAGCTGAACAAACGTGTGGGTCAC
GTTTATAAGAAAGCGTTTATGCAAGTGGTTGAAGCGGACATGCTGAGC
AAAGGCCTGCTGGGTGGCGAGGCGTGGAGCAGCTGGCACAAAGAAGAT
AGCATCCACGTGGGTGTTCGTTGCATCGAGATGCTGATTGAAAGCACC
GGCATGGTGAGCCTGCACCGTCAGAACGCGGGCGTGGTTGGTCAAGAC
AGCGAAACCATCGAACTGGCGCCGGAGTACGCGGAAGCGATCGCGACC
CGTGCGGGCGCGCTGGCGGGTATCAGCCCGATGTTCCAGCCGTGCGTG
GTTCCGCCGAAACCGTGGACCGGTATTACCGGTGGCGGTTACTGGGCG
AACGGTCGTCGTCCGCTGGCGCTGGTGCGTACCCACAGCAAGAAAGCG
CTGATGCGTTACGAGGATGTTTATATGCCGGAAGTGTATAAGGCGATC
AACATTGCGCAAAACACCGCGTGGAAAATTAACAAGAAAGTGCTGGCG
GTTGCGAACGTGATCACCAAGTGGAAACACTGCCCGGTTGAGGACATC
CCGGCGATTGAACGTGAGGAACTGCCGATGAAGCCGGAGGACATCGAT
ATGAACCCGGAAGCGCTGACCGCGTGGCGTCGTGCGGCGGCGGCGGTG
TACCGTAAGGATAAAGCGCGCAAAAGCCGTCGTATTAGCCTGGAGTTC
ATGCTGGAACAGGCGAACAAGTTTGCGAACCACAAAGCGATCTGGTTC
CCGTACAACATGGACTGGCGTGGTCGTGTTTATGCGGTGCCAATGTTC
ACCCCGCAAGGCAACGATATGACCAAGGGTCTGCTGACCCTGGCGAAG
GGCAAACCGATTGGCAAAGAGGGTTACTATTGGCTGAAAATCCACGGC
GCGAACTGCGCGGGTGTTGACAAGGTGCCGTTCCCGGAGCGTATCAAG
TTCATCGAGGAAAACCACGAAAACATCATGGCGTGCGCGAAAAGCCCG
CTGGAGAACACCTGGTGGGCGGAACAGGATAGCCCGTTCTGCTTTCTG
GCGTTCTGCTTTGAATACGCGGGCGTTCAACACCACGGTCTGAGCTAT
AACTGCAGCCTGCCGCTGGCGTTTGATGGCAGCTGCAGCGGTATCCAG
CACTTCAGCGCGATGCTGCGTGATGAGGTTGGCGGTCGCGCGGTGAAC
CTGCTGCCGAGCGAAACCGTGCAGGACATCTACGGTATTGTTGCGAAG
AAAGTGAACGAGATCCTGCAAGCGGACGCGATTAACGGTACCGATAAC
GAGGTGGTTACCGTTACCGATGAAAACACCGGCGAGATCAGCGAAAAG
GTGAAACTGGGTACCAAGGCGCTGGCGGGCCAGTGGCTGGCGTATGGC
GTTACCCGTAGCGTGACCAAGCGTCCAGTTATGACCCTGGCGCTGGGC
AGCAAAGAGTTCGGTTTTCGTCAGCAAGTGCTGGAAGACACCATCCAA
CCGGCGATTGATAGCGGCAAGGGTCTGATGTTTACCCAGCCGAACCAA
GCGGCGGGTTACATGGCGAAACTGATCTGGGAGAGCGTGAGCGTTACC
GTGGTTGCGGCGGTTGAAGCGATGAACTGGCTGAAGAGCGCGGCGAAA
CTGCTGGCGGCGGAAGTGAAGGACAAGAAAACCGGTGAAATTCTGCGT
AAACGTTGCGCGGTTCACTGGGTGACCCCGGATGGCTTCCCGGTTTGG
CAGGAGTATAAGAAACCGATCCAAACCCGTCTGAACCTGATGTTCCTG
GGCCAGTTTCGTCTGCAACCGACCATCAACACCAACAAGGACAGCGAG
ATTGATGCGCACAAACAGGAAAGCGGTATTGCGCCGAACTTTGTGGCG
AGCCAAGACGGCAGCCACCTGCGTAAGACCGTGGTTTGGGCGCACGAG
AAATACGGTATCGAGAGCTTCGCGCTGATTCACGACAGCTTTGGTACC
ATCCCGGCGGATGCGGCGAACCTGTTCAAGGCGGTTCGTGAAACCATG
GTGGACACCTACGAAAGCTGCGATGTTCTGGCGGACTTCTATGATCAG
ATCGCGGACCAACTGCACGAAAGCCAGCTGGATAAAATGCCGGCGCTG
CCGGCGAAGGGTAACCTGAACCTGCGTGACATTCTGGAGAGCGATTAT
GCGTTTGCGTAA.

In some embodiments, the T7 RNA polymerase variant comprises a protein tag. Protein tags used are typically peptide sequences genetically grafted onto the protein. Protein tags may be attached to the N-terminus or the C-terminus of the T7 RNA polymerase variant. Protein tags may be removable by chemical agents or by enzymatic means, such as proteolysis or intein splicing.

In some embodiments, the protein tags may be an affinity tag which allows the polymerase variant to be purified from a crude biological source using an affinity technique. Such affinity tags may include, but are not limited to, albumin-binding protein (ABP), alkaline phosphatase (AP), AU1 epitope, AU5 epitope, bacteriophage T7 epitope (T7-tag), bacteriophage V5 epitope (V5-tag), Biotin-carboxy carrier protein (BCCP), Bluetongue virus tag (B-tag), Calmodulin binding peptide (CBP), Chloramphenicol Acetyl Transferase (CAT), Cellulose binding domain (CBP), chitin binding protein (CBP), Choline-binding domain (CBD), Dihydrofolate reductase (DHFR), E2 epitope, FLAG epitope, Galactose-binding protein (GBP), Green fluorescent protein (GFP), Glu-Glu (EE-tag), Glutathione S-transferase (GST), Human influenza hemagglutinin (HA), HaloTag®, Histidine affinity tag (HAT), Horseradish Peroxidase (HRP), HSV epitope, Ketosteroid isomerase (KSI), KT3 epitope, LacZ, Luciferase, Maltose-binding protein (MBP), Myc epitope, NusA, PDZ domain, PDZ ligand, Polyarginine (Arg-tag), Polyaspartate (Asp-tag), Polycysteine (Cys-tag), Polyhistidine (His-tag), Polyphenylalanine (Phe-tag), Profinity eXact, Protein C, S1-tag, S-tag, Streptavadin-binding peptide (SBP), Staphylococcal protein A (Protein A), Staphylococcal protein G (Protein G), Strep-tag, Streptavadin, Small Ubiquitin-like Modifier (SUMO), Tandem Affinity Purification (TAP), T7 epitope, Thioredoxin (Trx), TrpE, Ubiquitin, Universal, and VSV-G. Characteristics of these affinity tags have been described in the art, for example, in Kimple et al., Curr Protoc Protein Sci. 2013; 73: Unit 9.9.

In some embodiments, protein tags may be a solubilization tag which improves the solubility of the polymerase and/or prevents it from precipitating. Such solubilization tags may include, but not limited to, thioredoxin (Trx) and poly(NANP). Some affinity tags have a dual role as a solubilization agent, such as Maltose-binding protein (MBP), and Glutathione S-transferase (GST).

In some embodiments, protein tags may be a chromatography tag which alters chromatographic properties of the polymerase to afford different resolution across a particular separation technique. Such tags may comprise polyanionic amino acids, such as a FLAG-tag.

In some embodiments, the T7 RNA polymerase variant comprises a Polyhistidine (His-tag). It is well known to the skilled artisan that a His-tag is an amino acid sequence containing several, preferably 3 to 7, more preferred 6 consecutive histidines (hexa histidine-tag).

In some embodiments, the T7 RNA polymerase variant comprises a hexa histidine-tag.

In one embodiment, the T7 RNA polymerase variant comprises a hexa histidine-tag attached to the N-terminus of the polymerase variant.

In one embodiment, the T7 RNA polymerase variant comprises a hexa histidine-tag attached to the C-terminus of the polymerase variant.

In a His-tag sequence the Histidines represent the essential portion, but facultatively there may be a few additional amino acids comprised in the His-tag. For example, a N-terminal T7 RNA polymerase sequence including a His-tag may comprise the sequence MHHHHHHGS (SEQ ID NO.: 4). In the present exemplary His-tag the amino acids Glycine and Serine form a linker to the N-terminus of the T7 variant. The linker amino acids are part of the His-tag and typically arise as a cloning artifact of the nucleotide sequence encoding the His-tag. Typically, the linker sequence in the His-tag comprises 1 to 5 amino acids. However, in some cases, the linker sequence may include a sequence that allows the his-tag to be cleaved from the recombinant protein.

According to the disclosure, the N-terminal Methionine of a T7 RNA polymerase variant may be replaced by a His-tag. Alternatively, the N-terminal sequence of the T7 RNA polymerase variant according to the disclosure may be extended by the His-tag. In such a case, the N-terminus of the primary translation product of the T7 RNA polymerase variant comprises a N-terminal Methionine followed by the His-tag, followed by the Methionine encoded by the start codon of the original T7 RNA polymerase encoding nucleotide sequence.

Purification of a His-tagged T7 RNA polymerase wild-type or variant protein may be performed with immobilized metal affinity chromatography. This method is a widely employed method to purify recombinant proteins containing a short affinity-tag consisting of Histidine residues (His-tag). Immobilized metal-affinity chromatography (described by Porath et al., Metal chelate affinity chromatography, a new approach to protein fabrication; Nature. 1975; 258(5536): 598-599) is based on the interaction between a transition metal ion (Co²⁺, Ni²⁺, Cui²⁺, Zn²⁺) immobilized on a particulate metal chelating affinity matrix and specific amino acid side chains. Histidine is the amino acid that exhibits the strongest interaction with immobilized metal ion matrices, as electron donor groups on the Histidine imidazole ring readily form coordination bonds with the immobilized transition metal.

In one embodiment, a T7 RNA polymerase variant including a His-tag comprises the amino acid sequence described below. This variant has 892 amino acids with a molecular weight of 99836.6 g/mol and a predicted isoelectric point (pI) of 7.29.

(SEQ ID NO.: 5)
MHHHHHHGSMNTINIAKNDFSDIELAAIPFNTLADHYGERLAREQLAL
EHESYEMGEARFRKMFERQLKAGEVADNAAAKPLITTLLPKMIARIND
WFEEVKAKRGKRPTAFQFLQEIKPEAVAYITIKTTLACLTSADNTTVQ
AVASAIGRAIEDEARFGRIRDLEAKHFKKNVEEQLNKRVGHVYKKAFM
QVVEADMLSKGLLGGEAWSSWHKEDSIHVGVRCIEMLIESTGMVSLHR
QNAGVVGQDSETIELAPEYAEAIATRAGALAGISPMFQPCVVPPKPWT
GITGGGYWANGRRPLALVRTHSKKALMRYEDVYMPEVYKAINIAQNTA
WKINKKVLAVANVITKWKHCPVEDIPAIEREELPMKPEDIDMNPEALT
AWRRAAAAVYRKDKARKSRRISLEFMLEQANKFANHKAIWFPYNMDWR
GRVYAVPMFTPQGNDMTKGLLTLAKGKPIGKEGYYWLKIHGANCAGVD
KVPFPERIKFIEENHENIMACAKSPLENTWWAEQDSPFCFLAFCFEYA
GVQHHGLSYNCSLPLAFDGSCSGIQHFSAMLRDEVGGRAVNLLPSETV
QDIYGIVAKKVNEILQADAINGTDNEVVTVTDENTGEISEKVKLGTKA
LAGQWLAYGVTRSVTKRPVMTLALGSKEFGFRQQVLEDTIQPAIDSGK
GLMFTQPNQAAGYMAKLIWESVSVTVVAAVEAMNWLKSAAKLLAAEVK
DKKTGEILRKRCAVHWVTPDGFPVWQEYKKPIQTRLNLMFLGQFRLQP
TINTNKDSEIDAHKQESGIAPNFVASQDGSHLRKTVVWAHEKYGIESF
ALIHDSFGTIPADAANLFKAVRETMVDTYESCDVLADFYDQIADQLHE
SQLDKMPALPAKGNLNLRDILESDYAFA.

A codon-optimized nucleic acid sequence encoding the T7 RNA polymerase variant including a His-tag is provided below:

(SEQ ID NO.: 6)
ATGCATCACCACCACCACCACGGATCAATGAACACAATAAACATAGCT
AAGAACGACTTTAGCGACATCGAACTGGCGGCGATTCCGTTTAACACC
CTGGCGGACCACTATGGCGAGCGTCTGGCGCGTGAACAGCTGGCGCTG
GAGCACGAAAGCTATGAGATGGGTGAAGCGCGTTTCCGTAAGATGTTT
GAGCGTCAACTGAAAGCGGGCGAAGTTGCGGACAACGCGGCGGCGAAG
CCGCTGATTACCACCCTGCTGCCGAAAATGATCGCGCGTATTAACGAT
TGGTTCGAGGAAGTTAAGGCGAAACGTGGCAAGCGTCCGACCGCGTTC
CAGTTTCTGCAAGAGATCAAGCCGGAAGCGGTGGCGTACATCACCATT
AAAACCACCCTGGCGTGCCTGACCAGCGCGGACAACACCACCGTTCAA
GCGGTTGCGAGCGCGATTGGTCGTGCGATTGAGGACGAAGCGCGTTTT
GGCCGTATTCGTGATCTGGAGGCGAAGCACTTCAAGAAAAACGTTGAG
GAACAGCTGAACAAACGTGTGGGTCACGTTTATAAGAAAGCGTTTATG
CAAGTGGTTGAAGCGGACATGCTGAGCAAAGGCCTGCTGGGTGGCGAG
GCGTGGAGCAGCTGGCACAAAGAAGATAGCATCCACGTGGGTGTTCGT
TGCATCGAGATGCTGATTGAAAGCACCGGCATGGTGAGCCTGCACCGT
CAGAACGCGGGCGTGGTTGGTCAAGACAGCGAAACCATCGAACTGGCG
CCGGAGTACGCGGAAGCGATCGCGACCCGTGCGGGCGCGCTGGCGGGT
ATCAGCCCGATGTTCCAGCCGTGCGTGGTTCCGCCGAAACCGTGGACC
GGTATTACCGGTGGCGGTTACTGGGCGAACGGTCGTCGTCCGCTGGCG
CTGGTGCGTACCCACAGCAAGAAAGCGCTGATGCGTTACGAGGATGTT
TATATGCCGGAAGTGTATAAGGCGATCAACATTGCGCAAAACACCGCG
TGGAAAATTAACAAGAAAGTGCTGGCGGTTGCGAACGTGATCACCAAG
TGGAAACACTGCCCGGTTGAGGACATCCCGGCGATTGAACGTGAGGAA
CTGCCGATGAAGCCGGAGGACATCGATATGAACCCGGAAGCGCTGACC
GCGTGGCGTCGTGCGGCGGCGGCGGTGTACCGTAAGGATAAAGCGCGC
AAAAGCCGTCGTATTAGCCTGGAGTTCATGCTGGAACAGGCGAACAAG
TTTGCGAACCACAAAGCGATCTGGTTCCCGTACAACATGGACTGGCGT
GGTCGTGTTTATGCGGTGCCAATGTTCACCCCGCAAGGCAACGATATG
ACCAAGGGTCTGCTGACCCTGGCGAAGGGCAAACCGATTGGCAAAGAG
GGTTACTATTGGCTGAAAATCCACGGCGCGAACTGCGCGGGTGTTGAC
AAGGTGCCGTTCCCGGAGCGTATCAAGTTCATCGAGGAAAACCACGAA
AACATCATGGCGTGCGCGAAAAGCCCGCTGGAGAACACCTGGTGGGCG
GAACAGGATAGCCCGTTCTGCTTTCTGGCGTTCTGCTTTGAATACGCG
GGCGTTCAACACCACGGTCTGAGCTATAACTGCAGCCTGCCGCTGGCG
TTTGATGGCAGCTGCAGCGGTATCCAGCACTTCAGCGCGATGCTGCGT
GATGAGGTTGGCGGTCGCGCGGTGAACCTGCTGCCGAGCGAAACCGTG
CAGGACATCTACGGTATTGTTGCGAAGAAAGTGAACGAGATCCTGCAA
GCGGACGCGATTAACGGTACCGATAACGAGGTGGTTACCGTTACCGAT
GAAAACACCGGCGAGATCAGCGAAAAGGTGAAACTGGGTACCAAGGCG
CTGGCGGGCCAGTGGCTGGCGTATGGCGTTACCCGTAGCGTGACCAAG
CGTCCAGTTATGACCCTGGCGCTGGGCAGCAAAGAGTTCGGTTTTCGT
CAGCAAGTGCTGGAAGACACCATCCAACCGGCGATTGATAGCGGCAAG
GGTCTGATGTTTACCCAGCCGAACCAAGCGGCGGGTTACATGGCGAAA
CTGATCTGGGAGAGCGTGAGCGTTACCGTGGTTGCGGCGGTTGAAGCG
ATGAACTGGCTGAAGAGCGCGGCGAAACTGCTGGCGGCGGAAGTGAAG
GACAAGAAAACCGGTGAAATTCTGCGTAAACGTTGCGCGGTTCACTGG
GTGACCCCGGATGGCTTCCCGGTTTGGCAGGAGTATAAGAAACCGATC
CAAACCCGTCTGAACCTGATGTTCCTGGGCCAGTTTCGTCTGCAACCG
ACCATCAACACCAACAAGGACAGCGAGATTGATGCGCACAAACAGGAA
AGCGGTATTGCGCCGAACTTTGTGGCGAGCCAAGACGGCAGCCACCTG
CGTAAGACCGTGGTTTGGGCGCACGAGAAATACGGTATCGAGAGCTTC
GCGCTGATTCACGACAGCTTTGGTACCATCCCGGCGGATGCGGCGAAC
CTGTTCAAGGCGGTTCGTGAAACCATGGTGGACACCTACGAAAGCTGC
GATGTTCTGGCGGACTTCTATGATCAGATCGCGGACCAACTGCACGAA
AGCCAGCTGGATAAAATGCCGGCGCTGCCGGCGAAGGGTAACCTGAAC
CTGCGTGACATTCTGGAGAGCGATTATGCGTTTGCGTAA.

T7 RNA polymerase variants of the present disclosure may have greater enzymatic activity in incorporating a 2′ modified (e.g., 2′-O-methyl) nucleotide than the enzymes that are currently available. For example, a T7 RNA polymerase variant of the present disclosure may have greater enzymatic activity in incorporating a 2′ modified (e.g., 2′-O-methyl) nucleotide than the wild-type T7 RNA polymerase.

For example, a T7 RNA polymerase variant of the present disclosure may be 1 to 100 times more active in incorporating a 2′ modified (e.g., 2′-O-methyl) nucleotide than the LA variant (Y639L, H784A; U.S. Pat. No. 8,105,813) under the same reaction conditions.

For example, a T7 RNA polymerase variant of the present disclosure may be 1 to 100 times more active in incorporating a 2′ modified (e.g., 2′-O-methyl) nucleotide than the LAR variant (Y639L, H784A, K378R; U.S. Pat. No. 8,105,813) under the same reaction conditions.

As yet another example, a T7 RNA polymerase variant of the present disclosure may be 1 to 10 times more active in incorporating a 2′ modified (e.g., 2′-O-methyl) nucleotide than the RGVG-M5 variant (S430P, N433T, E593G, S633P, Y639V, V685A, H784G, F849I, F880Y; Meyer et al., Transcription yield of fully 2′-modified RNA can be increased by the addition of thermostabilizing mutations to T7 RNA polymerase mutants; Nucleic Acids Res. 2015; 43(15):7480-7488; and U.S. Pat. No. 9,988,612) under the same reaction conditions.

In some embodiments, T7 RNA polymerase variants of the present disclosure may generate higher yields of 2′ modified (e.g., 2′-O-methyl) oligonucleotides than enzymes that are currently available under the same reaction conditions. For example, a T7 RNA polymerase variant of the present disclosure may generate higher yields of 2′ modified (e.g., 2′-O-methyl) oligonucleotides than the wild-type T7 RNA polymerase under the same reaction conditions.

For example, a T7 RNA polymerase variant described herein may generate 1 to 100 folds more yields of 2′ modified oligonucleotides than the LA variant (Y639L, H784A; U.S. Pat. No. 8,105,813) under the same reaction conditions.

For example, a T7 RNA polymerase variant of the present disclosure may generate 1 to 100 folds more yields of 2′ modified oligonucleotides than the LAR variant (Y639L, H784A, K378R; U.S. Pat. No. 8,105,813) under the same reaction conditions.

As yet another example, a T7 RNA polymerase variant of the present disclosure may generate 1 to 10 folds more yields of 2′ modified oligonucleotides than the RGVG-M5 variant (S430P, N433T, E593G, S633P, Y639V, V685A, H784G, F849I, F880Y; Meyer et al., Nucleic Acids Res. 2015; 43(15):7480-7488; and U.S. Pat. No. 9,988,612) under the same reaction conditions.

In some embodiments, a T7 RNA polymerase variant of the present disclosure used with the transcription methods of the disclosure does not require the presence of 2′-OH GTP.

In some embodiments, a T7 RNA polymerase variant of the present disclosure used with the transcription methods of the disclosure does not require the presence of GMP (guanosine monophosphate) and still produces the same yield.

In some embodiments, a T7 RNA polymerase variant of the present disclosure does not have a bias in incorporating a 2′-modified ATP, CTP, UTP, or GTP. In one embodiment, a T7 RNA polymerase variant of the present disclosure does not have a bias in incorporating 2′-O-methyl ATP, 2′-O-methyl CTP, 2′-O-methyl UTP, or 2′-O-methyl GTP.

In some embodiments, a T7 RNA polymerase variant of the present disclosure may have enhanced thermostability than the wild-type T7 RNA polymerase.

In some embodiments, a T7 RNA polymerase variant of the present disclosure is capable of producing an oligonucleotide at an elevated temperature. For example, a T7 polymerase variant may be used to synthesize an oligonucleotide of interest at a temperature of 45-50° C.

### Transcription Methods

In some embodiment, the present disclosure provides methods and conditions for using polymerase variants described herein to enzymatically incorporate modified nucleotides into oligonucleotides.

In some embodiments, the transcription methods utilize a polymerase variant that is capable of incorporating a 2′-modified nucleotide as described herein.

In one embodiment, the transcription methods utilize a polymerase variant that is LAR-M5 (SEQ ID NO.: 2). In some embodiments, the transcription methods utilize a polymerase variant that is LAR-M5 with a His-tag (SEQ ID NO.: 5).

In further embodiments, the transcription methods utilize a polymerase variant that is LAR-M6 or LAR-M6 with a His-tag.

In some embodiments, the concentration of the polymerase variant may range from 0.2 ng/μl to 2000 ng/μL, or from 0.2 ng/μl to 1000 ng/μL, or from 1 ng/μl to 1000 ng/μl, from 1 ng/μl to 500 ng/μl, or from 1 ng/μl to 100 ng/μl, or from 10 ng/μl to 50 ng/μl, or 10 ng/μl, or 20 ng/μl, or 30 ng/μl, or 40 ng/μl, or 50 ng/μl. In one embodiment, the concentration of the polymerase variant is 25 ng/μl. In one embodiment, the concentration of the polymerase variant is 30 ng/μL.

In some embodiments, a T7 RNA polymerase variant of the present disclosure may be used with a transcription mixture containing 2′-O-methyl A, G, C, and U nucleoside triphosphates.

In some embodiments, a T7 RNA polymerase variant of the present disclosure may be used with a transcription mixture containing 2′-fluoro A, G, C, and U nucleoside triphosphates.

In some embodiments, a T7 RNA polymerase variant of the present disclosure may be used with a transcription mixture containing 2′-amino A, G, C, and U nucleoside triphosphates.

In some embodiments, a T7 RNA polymerase variant of the present disclosure may be used with a transcription mixture containing one or more of the following nucleotides: 2′-O-methyl ATP, 2′-O-methyl CTP, 2′-O-methyl UTP, 2′-O-methyl GTP, 2′-fluoro ATP, 2′-fluoro CTP, 2′-fluoro UTP, 2′-fluoro GTP, 2′-amino ATP, 2′-amino CTP, 2′-amino UTP, and 2′-amino GTP, and combinations thereof.

In some embodiments, a T7 RNA polymerase variant of the present disclosure may be used with a transcription mixture containing 2′-azido A, G, C, and U nucleoside triphosphates.

In further embodiments, a T7 RNA polymerase variant of the present disclosure may be used with an rRmY, dRmY, rGmH, fGmH, dGmH, dAmB, rRdY, dRdY or rN transcription mixture.

As used herein, a transcription mixture containing only 2′-O-methyl A, G, C, and U nucleoside triphosphates is referred to as an MNA mixture, and oligonucleotides produced therefrom are referred to as MNA oligonucleotides and contains only 2′-O-methyl nucleotides. A transcription mixture containing 2′-OMe C and U and 2′-OH A and G is referred to as an “rRmY” mixture and oligonucleotides produced therefrom are referred to as “rRmY” oligonucleotides. A transcription mixture containing deoxy A and G and 2′-OMe U and C is referred to as a “dRmY” mixture and oligonucleotides produced therefrom are referred to as “dRmY” oligonucleotides. A transcription mixture containing 2′-OMe A, C, and U, and 2′-OH G is referred to as a “rGmH” mixture and oligonucleotides produced therefrom are referred to as “rGmH” oligonucleotides. A transcription mixture containing 2′-OMe A, U, and C, and 2′-F G is referred to as a “fGmH” mixture and oligonucleotides produced therefrom are referred to as “fGmH” oligonucleotides. A transcription mixture containing 2′-OMe A, U, and C, and deoxy G is referred to as a “dGmH” mixture and oligonucleotides produced therefrom are referred to as “dGmH” oligonucleotides. A transcription mixture containing deoxy A, and 2′-OMe C, G and U is referred to as a “dAmB” mixture and oligonucleotides produced therefrom are referred to as “dAmB” oligonucleotides. A transcription mixture containing 2′-OH A and 2′-OMe C, G and U is referred to as a “rAmB” mixture and oligonucleotides produced therefrom are referred to as “rAmB” oligonucleotides. A transcription mixture containing 2′-OH adenosine triphosphate and guanosine triphosphate and deoxy cytidine triphosphate and thymidine triphosphate is referred to as an “rRdY” mixture and oligonucleotides produced therefrom are referred to as “rRdY” oligonucleotides. A transcription mixture containing all 2′-OH nucleotides is referred to as a “rN” mixture and oligonucleotides produced therefrom are referred to as “rN”, “rRrY” or RNA oligonucleotides, and a transcription mixture containing all deoxy nucleotides is referred to as a “dN” mixture and oligonucleotides produced therefrom are referred to as “dN” or “dRdY” or DNA oligonucleotides.

The modified NTP (e.g., 2′-OMe NTP) concentration (each NTP) may range from 5 μM to 5 mM, or from 5 μM to 1 mM, or from 50 μM to 1 mM, or from 0.1 mM to 5 mM, or from 0.1 mM to 2 mM. In one embodiment, the concentration of the modified NTPs (e.g., 2′-OMe NTP) is 0.5 mM for each NTP.

A number of factors may be necessary for the transcription conditions useful in the methods described herein.

The transcription conditions may require a DNA template. In some embodiments, the DNA template may be present in a concentration of about 5 nM to 500 nM, or about 10 nM to 500 nM, or about 10 nM to 400 nM, or about 50 nM to 400 nM. In some embodiments, the DNA template may be present in a concentration of about 50 nM, 55 nM, 60 nM, 65 nM, 70 nM, 75 nM, 80 nM, 85 nM, 90 nM, 95 nM, 100 nM, 110 nM, 120 nM, 130 nM, 140 nM, 150 nM, 200 nM, 250 nM, 300 nM, 350 nM, or 400 nM. In one embodiment, the DNA template concentration is about 100 nM. In another embodiment, the DNA template concentration is about 200 nM. Additionally, increases in the yields of modified transcripts may be observed when a leader sequence is incorporated into the 5′ end of a fixed sequence at the 5′ end of the DNA transcription template, such that at least about the first 6 residues of the resultant transcript are all purines.

Another factor in the incorporation of 2′-O-methyl substituted nucleotides into transcripts is the use of divalent metal ions such as magnesium and manganese in the transcription mixture. As is known in the art, different combinations of concentrations of magnesium chloride and manganese chloride affect yields of 2′-O-methylated transcripts, with the optimum concentration of the magnesium and manganese chloride being dependent on the concentration in the transcription reaction mixture of NTPs which complex divalent metal ions. For example, to obtain the greatest yields of maximally 2′ substituted O-methylated transcripts, concentrations of approximately 5 or 6 mM magnesium chloride and 1.5 mM manganese chloride may be used when each NTP is present at a concentration of 0.5 mM. When the concentration of each NTP is 1.0 mM, concentrations of approximately 6.5 mM magnesium chloride and 2.0 mM manganese chloride may be used. When the concentration of each NTP is 2.0 mM, concentrations of approximately 9.5 mM magnesium chloride and 3 mM manganese chloride may be used.

However, in each case, departures from these concentrations of up to two-fold still give significant amounts of modified transcripts. More broadly, the magnesium chloride concentration may range from 0.5 mM to 50 mM. The manganese chloride concentration may range from 0.15 mM to 15 mM. Both magnesium chloride and manganese chloride are preferred to be present within the ranges described and in a preferred embodiment are present in about a 10 to about 3 ratio of magnesium chloride:manganese chloride, preferably, the ratio is about 3-5:1, and more preferably, the ratio is about 3-4:1.

Those skilled in the art will recognize that magnesium chloride may be replaced by magnesium acetate, and manganese chloride may be replaced by manganese acetate. Similarly, the magnesium acetate concentration may range from 0.5 mM to 50 mM. The manganese acetate concentration may range from 0.15 mM to 15 mM. Both magnesium acetate and manganese acetate are preferred to be present within the ranges described and in a preferred embodiment are present in about a 10 to about 3 ratio of magnesium acetate:manganese acetate, preferably, the ratio is about 3-5:1, and more preferably, the ratio is about 3-4:1. In one embodiment, the concentration of magnesium acetate is about 6 mM and the concentration of manganese acetate is about 1.5 mM.

Another factor in obtaining transcripts incorporating modified nucleotides is the presence or concentration of 2′-OH GTP. Transcription can be divided into two phases: the first phase is initiation, during which an NTP is added to the 3′-hydroxyl end of GTP (or another substituted guanosine) to yield a dinucleotide which is then extended by about 10-12 nucleotides, the second phase is elongation, during which transcription proceeds beyond the addition of the first about 10-12 nucleotides. It has been found that small amounts of 2′-OH GTP added to a transcription mixture containing an excess of 2′-OMe GTP are sufficient to enable the polymerase to initiate transcription using 2′-OH GTP, but once transcription enters the elongation phase the reduced discrimination between 2′-OMe and 2′-OH GTP, and the excess of 2′-OMe GTP over 2′-OH GTP allows the incorporation of primarily the 2′-OMe GTP.

However, in some embodiments, a T7 RNA polymerase variant of the present disclosure used with the transcription methods of the disclosure does not require the presence of 2′-OH GTP.

In some embodiments, the 2′-OH GTP concentration may range from 0 μM to 300 μM. In some embodiments, the transcription reaction does not require the addition of 2′-OH GTP.

Priming transcription with GMP (guanosine monophosphate) or guanosine may also be used. This effect results from the specificity of the polymerase for the initiating nucleotide. As a result, the 5′-terminal nucleotide of any transcript generated in this fashion is likely to be 2′-OH G. However, in some cases, a T7 RNA polymerase variant of the present disclosure does not require GMP and still produces the same yield.

In some embodiments, the 2′-OH GMP concentration may range from 0 to 5 mM. The concentration of GMP (or guanosine) may be 0.5 mM and more preferably at 1 mM. In one embodiment, the concentration of GMP is about 1 mM. In other embodiments, the transcription reaction does not contain GMP.

Additionally, it has also been found that including polyethylene glycol (PEG), preferably PEG-8000, in the transcription reaction is useful to improve incorporation of modified nucleotides. The PEG-8000 concentration may range from 0 to 50% (w/v), or from 1 to 50% (w/v), or from 5 to 20% (w/v). In one embodiment, the PEG-8000 concentration is about 5% (w/v), 6% (w/v), 7% (w/v), 8% (w/v), 9% (w/v), 10% (w/v), 11% (w/v), 12% (w/v), 13% (w/v), 14% (w/v), 15% (w/v), 16% (w/v), 17% (w/v), 18% (w/v), 19% (w/v), or 20% (w/v). In one embodiment, the PEG-8000 concentration is about 10% (w/v). The methods of the present disclosure also include the use of other hydrophilic polymer including, for example, other molecular weight PEG or other polyalkylene glycols.

Besides the factors described above, the transcription reaction may further comprise one or more of the following components: a buffering agent (e.g., HEPES), a reducing agent (e.g., Dithiothreitol (DTT)), a polyamine (e.g., spermidine or spermine), a detergent (e.g., Triton X-100), and inorganic pyrophosphatase. Exemplary ranges of such components in the transcription conditions are described below:

A buffering agent such as HEPES (4-(2-hydroxyethyl)-1-piperazineethanesulfonic acid) is used to provide a pH suitable for the enzymatic reaction. The concentration of the buffering agent (e.g., HEPES) may range from 0 to 1 M. In one embodiment, the concentration of the buffering agent (e.g., HEPES) is about 200 mM. The transcription mixture may include the use of other buffering agents that have a pKa between 5 and 10, including, for example, Tris-hydroxymethyl-aminomethane.

A reducing agent such as DTT (Dithiothreitol) or βME (2-mercaptoethanol) may be added. The DTT concentration may range from 0 to 400 mM. In one embodiment, the DTT concentration is about 40 mM.

The polyamine, such as spermidine and/or spermine, may have a concentration ranging from 0 to 20 mM. In one embodiment, the polyamine (e.g., spermidine) concentration is about 2 mM.

The Triton X-100 concentration may range from 0 to 0.1% (w/v). In one embodiment, the concentration of Triton X-100 is about 0.01% (w/v). The methods of the present disclosure also provide for the use of other non-ionic detergents including, for example, other detergents, including other Triton-X detergents.

Inorganic pyrophosphatase catalyzes the hydrolysis of inorganic pyrophosphate produced from the transcription. The addition of inorganic pyrophosphatase in the transcription reaction may prevent the accumulation of pyrophosphate and increase the yields of the transcription reaction. The inorganic pyrophosphatase can range from 0 to 100 units/ml. As used herein, one unit of inorganic pyrophosphatase is defined as the amount of enzyme that will liberate 1.0 mole of inorganic orthophosphate per minute at pH 7.2 and 25° C. In one embodiment, the concentration of the inorganic pyrophosphatase is about 5 units/ml.

The methods of the present disclosure can be practiced within the pH range of activity of most polymerases that incorporate modified nucleotides. For example, the pH of the transcription mixture may range from pH 6 to pH 9. In one embodiment, the pH is about 7.5.

In addition, the methods of the present disclosure provide for the optional use of chelating agents in the transcription reaction condition including, for example, EDTA, EGTA, and DTT.

The transcription reaction may be performed at normal temperature (e.g., about 37-41° C.). The transcription reaction may also be performed at an elevated temperature such as about 45 to about 50° C. Performing transcription reactions at elevated temperatures may improve the reaction kinetics and lead to higher yields of modified transcripts.

The transcription reaction may be allowed to occur from about one hour to weeks, e.g., from about 1 to about 24 hours.

As a non-limiting example for synthesizing a 2′-O-methylated oligonucleotide, the transcription reaction may contain 200 mM HEPES/KOH pH 7.5, 6 mM Mg(OAc)₂, 1.5 mM Mn(OAc)₂, 2 mM spermidine, 0.5 mM each 2′-O-methyl-NTP, 40 mM DTT, 0.01% (w/v) Triton X-100, 10% (w/v) PEG-8000, 5 units/ml inorganic pyrophosphatase, 1 mM GMP, 200 nM DNA template, and 30 ng/μ1 T7 RNA polymerase variant. The reaction may be carried out at 37° C. from about 1 to 24 hours.

In some embodiments, the polymerase variants of the present disclosure may be used in the SELEX™ method. The process of performing the SELEX™ method is detailed in the PCT Patent Publication No. WO2005111238. In particular, the polymerase variants of the present disclosure may be used in the 2′ modified SELEX™ process. The SELEX™ method used to generate 2′-modified aptamers is described, e.g., in U.S. Provisional Patent Application Ser. No. 60/430,761, filed Dec. 3, 2002, U.S. Provisional Patent Application Ser. No. 60/487,474, filed Jul. 15, 2003, U.S. Provisional Patent Application Ser. No. 60/517,039, filed Nov. 4, 2003, U.S. patent application Ser. No. 10/729,581, filed Dec. 3, 2003, and U.S. patent application Ser. No. 10/873,856, filed Jun. 21, 2004, entitled “Method for in vitro Selection of 2′-O-methyl Substituted Nucleic Acids”; the contents of each of which are herein incorporated by reference in their entirety.

The transcription products can be used as the library in the SELEX™ process to identify aptamers and/or to determine a conserved motif of sequences that have binding specificity to a given target. The resulting sequences are already partially stabilized, eliminating this step from the process to arrive at an optimized aptamer sequence and giving a more highly stabilized aptamer as a result. Another advantage of the 2′-OMe SELEX™ process is that the resulting sequences are likely to have fewer 2′-OH nucleotides required in the sequence, possibly none. To the extent 2′-OH nucleotides remain they can be removed by performing post-SELEX™ modifications.

### Modified Polynucleotides

In some embodiments, the present disclosure provides modified polynucleotides generated by the materials, reagents and methods described herein.

The term “polynucleotide”, as used herein, refers to a polymeric form of nucleotides or nucleotide analogs of any length, either deoxyribonucleotides or ribonucleotides, or modified nucleotides thereof, or mixtures thereof. The term “oligonucleotide”, as used herein, refers to a polynucleotide comprising from about 2 to about 300 nucleotides. In such cases, the terms “polynucleotide” and “oligonucleotide” are used interchangeably.

The term “nucleic acid”, in its broadest sense, includes any compound and/or substance that comprise a polymer of nucleotides. These polymers are often referred to as polynucleotides. The term “polynucleotide”, as used herein, refers to a polymeric form of nucleotides or nucleotide analogs of any length, either deoxyribonucleotides or ribonucleotides, or analogs thereof, or mixtures thereof. Exemplary nucleic acid molecules or polynucleotides of the disclosure include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

The skilled artisan will recognize that the term “RNA molecule” or “ribonucleic acid molecule” encompasses not only RNA molecules as expressed or found in nature, but also analogs and derivatives of RNA comprising one or more ribonucleotide/ribonucleoside analogs or derivatives as described herein or as known in the art. Strictly speaking, a “ribonucleoside” includes a nucleoside base and a ribose sugar, and a “ribonucleotide” is a ribonucleoside with one, two or three phosphate moieties. However, the terms “ribonucleoside” and “ribonucleotide” can be considered to be equivalent as used herein. The RNA can be modified in the nucleobase structure, the ribofuranosyl ring or in the ribose-phosphate backbone.

Modified oligonucleotides may be synthesized entirely of modified nucleotides, or with a subset of modified nucleotides. All nucleotides may be modified, and all may contain the same modification. All nucleotides may be modified, but contain different modifications, e.g., all nucleotides containing the same base may have one type of modification, while nucleotides containing other bases may have different types of modification. All purine nucleotides may have one type of modification (or are unmodified), while all pyrimidine nucleotides have another, different type of modification (or are unmodified). In this manner, transcripts, or pools of transcripts are generated using any combination of modifications, including for example, ribonucleotides (2′-OH), deoxyribonucleotides (2′-deoxy), 2′-F, and 2′-OMe nucleotides. Additionally, modified oligonucleotides may contain nucleotides bearing more than one modification simultaneously such as a modification at the internucleotide linkage (e.g., phosphorothioate) and at the sugar (e.g., 2′-OMe) and the base (e.g., inosine).

In some embodiments, modified oligonucleotides provided herein are 2′-OMe modified oligonucleotides. In a non-limiting example, the modified oligonucleotide is fully 2′-OMe modified.

In some embodiments, the modified oligonucleotide may be a nucleic acid aptamer, a ribozyme, an siRNA, a pre-miRNA, a miRNA, or an antisense RNA. In one embodiment, the modified oligonucleotide is a modified nucleic acid aptamer.

### Nucleic Acid Aptamers

In some embodiments, the present disclosure provides nucleic acid aptamers produced by the materials and methods described herein. In some examples, the nucleic acid aptamers are modified for increased stability, specifically with increased stability in vivo.

As used herein, an “aptamer” is a biomolecule that binds to a specific target molecule and modulates the target's activity, structure, or function. An aptamer may be nucleic acid or amino acid based. Aptamers, often called “chemical antibodies,” have similar characteristics as antibodies. In the context of the present disclosure, aptamers are nucleic acid aptamers. Nucleic acid aptamers comprise a series of linked nucleosides or nucleotides. A typical nucleic acid aptamer is approximately 10-15 kDa in size (20-45 nucleotides), binds its target with sub-nanomolar affinity, and discriminates against closely related targets. Nucleic acid aptamers have specific binding affinity to molecules through interactions other than classic Watson-Crick base pairing. Nucleic acid aptamers, like peptides generated by phage display or monoclonal antibodies (mAbs), are capable of specifically binding to selected targets and, through binding, block their targets' ability to function. A target of a nucleic acid aptamer may be but is not limited to, a protein, a nucleic acid molecule, a peptide, a small molecule and a whole cell.

Nucleic acid aptamers may be ribonucleic acid (RNA), deoxyribonucleic acid (DNA), or mixed ribonucleic acid and deoxyribonucleic acid (DNA/RNA hybrid). Aptamers may be single stranded ribonucleic acid, deoxyribonucleic acid or mixed ribonucleic acid and deoxyribonucleic acid.

Nucleic acid aptamers produced by the materials and methods described herein comprise at least one modified nucleotide e.g., a 2′-modified nucleotide. Such modification may increase the stability of an aptamer, e.g., by increasing its resistance to degradation by ribonucleases (RNases) present in a cell, thereby causing its half-life in the cell to be increased. The resistance of these modified aptamers to nuclease can be tested by incubating them with either purified nucleases or nuclease from mouse serum, and the integrity of aptamers can be analyzed by gel electrophoresis.

Modifications may also or alternatively be used to decrease the likelihood or degree to which aptamer introduced into cells elicit innate immune responses. Such responses, which have been well characterized in the context of RNA interference (RNAi), including small-interfering RNAs (siRNAs), as described in the art, tend to be associated with reduced half-life of the RNA and/or the elicitation of cytokines or other factors associated with immune responses.

The nucleic acid aptamers produced by the materials and methods described herein may have increased thermostability due to the incorporation of the modified nucleosides. Thermostability of nucleic acid aptamers is an important factor that controls the structure, hybridization, and functions of aptamers. The presence of 2′-O-Methyl nucleosides in DNA or RNA strands enhances thermostability of the duplexes and the effect is more prominent in RNA:RNA duplexes than in RNA:DNA duplexes. Modification of the 2′-deoxyribose with 2′-fluoro enhances the thermostability of the DNA-DNA duplexes by 1.3° C. per insertion.

In some embodiments, such modified nucleic acid aptamers may be synthesized entirely of modified nucleotides, or with a subset of modified nucleotides using the materials and methods described herein. The modifications can be the same or different. All nucleotides may be modified, and all may contain the same modification. All nucleotides may be modified, but contain different modifications, e.g., all nucleotides containing the same base may have one type of modification, while nucleotides containing other bases may have different types of modification. For example, all purine nucleotides may have one type of modification (or are unmodified), while all pyrimidine nucleotides have another, different type of modification (or are unmodified). In this way, oligonucleotides, or libraries of oligonucleotides are generated using any combination of modifications as disclosed herein.

The nucleic acid aptamers may comprise other modifications including, but not limited to, for example, (a) end modifications, e.g., 5′ end modifications (phosphorylation, dephosphorylation, conjugation, inverted linkages, etc.), 3′ end modifications (conjugation, DNA nucleotides, inverted linkages, etc.), (b) sugar modifications (e.g., at the 2′ position or 4′ position) or replacement of the sugar, (c) base modifications, e.g., replacement with modified bases, stabilizing bases, destabilizing bases, or bases that base pair with an expanded repertoire of partners, or conjugated bases, as well as (d) internucleoside linkage modifications, including modification or replacement of the phosphodiester linkages. Specific examples of modified aptamer compositions useful with the methods described herein include, but are not limited to, nucleic acid molecules containing modified or non-natural internucleoside linkages. Modified aptamer having modified internucleoside linkages include, among others, those that do not have a phosphorous atom in the internucleoside linkage. In other embodiments, modified aptamer has a phosphorus atom in its internucleoside linkage(s).

In some embodiments, the chemical modification is selected from a chemical substitution of the nucleic acid at a sugar position, a chemical substitution at a phosphate position and a chemical substitution at a base position. In other embodiments, the chemical modification is selected from incorporation of a modified nucleotide; 3′ capping; 5′ capping; conjugation to a high molecular weight, non-immunogenic compound; conjugation to a lipophilic compound; and incorporation of phosphorothioate into the phosphate backbone.

In some embodiments, nucleic acid aptamers of the present disclosure may be further modified by any number of conjugates. As a non-limiting example, the conjugates may be high molecular weight non-immunogenic compounds. In one embodiment, the high molecular weight, non-immunogenic compound is polyalkylene glycol, and more preferably is polyethylene glycol (PEG).

As non-limiting examples, a nucleic acid aptamer can also include at least one modified ribonucleoside including but not limited to a 2′-O-methyl modified nucleoside, a nucleoside comprising a 5′ phosphorothioate group, a terminal nucleoside linked to a cholesteryl derivative or dodecanoic acid bisdecylamide group, a locked nucleoside (e.g., β-D-ribo configuration, α-LNA having an α-L-ribo configuration (a diastereomer of LNA), 2′-amino-LNA having a 2′-amino functionalization, and 2′-amino-α-LNA having a 2′-amino functionalization), an abasic nucleoside, an inverted deoxynucleoside or inverted ribonucleoside, a 2′-deoxy-2′-fluoro modified nucleoside, a 2′-amino-modified nucleoside, a 2′-alkyl-modified nucleoside, a 2′-O-alkyl-modified nucleoside, a 2′-O-alkyl-O-alkyl-modified nucleoside, a 2′-fluoro-modified nucleoside, a 2′-fluoro-modified nucleoside, morpholino nucleoside, a phosphoramidate or a non-natural base comprising nucleoside, or any combination thereof. Alternatively, a nucleic acid aptamer can comprise at least two modified ribonucleosides, at least 3, at least 4, at least 5, at least 6, at least 7, at least 8, at least 9, at least 10, at least 15, at least 20 or more, up to the entire length of the molecule. The modifications need not be the same for each of such a plurality of modified deoxy- or ribonucleosides in a nucleic acid molecule.

Nucleic acid aptamers described herein may also contain one of the following at the 2′ position: H (deoxyribose); OH (ribose); F; O-, S-, or N-alkyl; O-, S-, or N-alkenyl; 0-, S- or N-alkynyl; or O-alkyl-O-alkyl, wherein the alkyl, alkenyl and alkynyl can be substituted or unsubstituted C1 to C10 alkyl or C2 to C10 alkenyl and alkynyl. Exemplary modifications include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In some embodiments, nucleic acid aptamers include one of the following at the 2′ position: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, a reporter group, an intercalator, a group for improving the pharmacokinetic properties of an aptamer, or a group for improving the pharmacodynamic properties of a nucleic acid aptamer, and other substituents having similar properties. In some embodiments, the modification includes a 2′ methoxyethoxy (2′-O—CH₂CH₂OCH₃, also known as 2′-O-(2-methoxyethyl) or 2′-MOE) (Martin et al., Helv. Chim. Acta, 1995; 78: 486-504) i.e., an alkoxy-alkoxy group. Another exemplary modification is 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Similar modifications may also be made at other positions on the aptamer, particularly the 3′ position of the sugar on the 3′ terminal nucleotide and the 5′ position of 5′ terminal nucleotide. Aptamers may also have sugar mimetics, such as cyclobutyls in place of the pentofuranosyl group. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference in its entirety.

Additional modifications which may be useful in the nucleic acid aptamers of the disclosure include those taught in, for example, International Publication PCT/US2012/058519, the contents of which are incorporated herein by reference in their entirety.

A suitable nucleotide length for an aptamer ranges from about 15 to about 100 nucleotide (nt), and in various other preferred embodiments, 15-30 nt, 20-25 nt, 30-100 nt, 30-60 nt, 25-70 nt, 25-60 nt, 40-60 nt, 25-40 nt, 30-40 nt, any of 22, 23, 24, 25, 26, 27, 28, 29, 30, 31, 32, 33, 34, 35, 36, 37, 38, 39 or 40 nt or 40-70 nt in length. However, the sequence can be designed with sufficient flexibility such that it can accommodate interactions of aptamers with their targets.

In some embodiments, the nucleic acid aptamer comprises one or more regions of double stranded character. Such double stranded regions may arise from internal self-complementarity or complementarity with a second or further aptamer molecule. In some embodiments, the double stranded region may be from 4-12, 4-10, 4-8 base pairs in length. In some embodiments, the double stranded region may be 5, 6, 7, 8, 9, 10, 11 or 12 base pairs. In some embodiments, the double stranded region may form a stem region. Such extended stem regions having double stranded character can serve to stabilize the nucleic acid aptamer. Extended stem regions of at least 6 base pairs may contribute to greater thermostability at higher temperatures and thereby, greater affinity for a target and increased overall functionality as compared to shorter stem regions of 5 base pairs or less. Shorter stem regions may lead to unraveling of the stem-loop structure at higher temperatures and associated loss of affinity and overall functionality.

Nucleic acid aptamers of the present disclosure may bind to a target with high binding affinity and specificity. As used herein, the term “target” refers to any molecule to which an aptamer can bind. The target can be a protein, a polypeptide, a peptide, a nucleic acid, a polysaccharide, a lipid molecule, or a chemical compound. The targets may be receptors, hormones, toxins, antigens, allergens, antibodies, pathogens (viruses, bacteria), metabolites, cofactors, inhibitors, activators, drugs, growth factors, enzymes, cells and tissues. In one embodiment, the target is a protein. As used herein, the term “binding affinity” refers to the binding characteristics, in particular the binding affinity of a given ligand, e.g., aptamer, that can be determined by methods known to those skilled in the art, such as enzymatic assays and the like.

### Kits

Also provided herein are kits for using the materials and/or practicing the methods of the present disclosure. Such kits according to the present disclosure may comprise at least a T7 RNA polymerase variant. The T7 RNA polymerase variant may be provided in a stable storage solution. Alternatively, such kits may comprise a nucleic acid molecule encoding the T7 RNA polymerase variant, an expression vector comprising a nucleic acid molecule encoding the T7 RNA polymerase variant, or a cell comprising an expression vector capable of expressing the T7 RNA polymerase variant. In such cases, the kit may provide reagents or materials for expressing and/or purifying the T7 RNA polymerase variant.

The kit of the disclosure may further comprise all or part, preferably all, of the reagents, factors, additives and/or oligonucleotide sequences which are necessary for carrying out the methods of the disclosure. For example, components provided in the kit may include: 1) nucleic acid templates and/or primers for generating the oligonucleotide of interest; 2) modified NTPs (either premixed or separate), for example, 2′-O-methyl NTPs, 2′-fluoro NTPs, and 2′-amino NTPs; 3) various components necessary for the enzymatic reaction (either premixed or separate), e.g., MgCl₂ (or Mg(OAc)₂), MnCl₂ (or Mn(OAc)₂), DTT, PEG-8000, GMP, buffering agent, polyamine, inorganic pyrophosphatase, etc.; and 4) purification reagents and components, like spin columns, etc.

The present disclosure is further illustrated by the following non-limiting examples.

### Examples

### Example 1. Preparation of T7 RNA Polymerase Variants

The DNA sequences of the T7 RNA polymerase variants, LAR-M5, LAR-M6 and RGVG-M5, were designed by incorporating specific point mutations at indicated positions (see Table 1) relative to the wild type T7 RNA polymerase sequence (SEQ ID NO: 1) deposited in the GenBank (NCBI Accession No: NP 041960.1). The sequences were codon optimized for E. coli expression and included with a His-tag encoding sequence at the N-terminus. After confirming by DNA sequencing, the sequences were sub-cloned into the plasmid pET30a for E. coli expression. The recombinant constructs were amplified and purified using standard molecular biology protocol.

To evaluate protein expression, E. coli BL21 Star (DE3) strain was transformed with a recombinant construct. A single colony was inoculated into Luria-Bertani (LB) medium containing the antibiotic kanamycin for selection. The cultures were incubated at 37° C. in a shaker incubator at 200 rotations per minute (rpm). Once the cell density reached an OD₆₀₀ (i.e., optical density measured at a wavelength of 600 nM) of 0.6-0.8, 0.5 mM Isopropyl β-D-1-thiogalactopyranoside (IPTG) was introduced to induce protein expression. The expression of the target protein was analyzed with sodium dodecyl sulfate-polyacrylamide gel electrophoresis (SDS-PAGE).

For purification, E. coli BL21 Star (DE3) cells harboring a recombinant construct was chosen for one-liter expression and purification. After induction with 0.5 mM IPTG at 37° C. for 4 h, cells were lysed in a lysis buffer containing 50 mM Tris-HCl pH 8.0, 150 mM NaCl, and 10% glycerol by sonication and cell debris pelleted by centrifugation. The supernatant after centrifugation was loaded onto a Ni-IDA column (GenScript) to retain the His-tagged protein. The target protein was eluted with a stepwise gradient of imidazole. Fractions were analyzed by SDS-PAGE. The fractions containing the target protein were pooled and concentrated, then loaded onto a Superdex® 200 size-exclusion column (GE Healthcare) for further purification. The samples were eluted in an elution buffer containing 50 mM Tris-HCl pH 8.0. SDS-PAGE was used to analyze the eluted fractions. The fractions containing the target protein were pooled and dialyzed into a dialysis buffer containing 50 mM Tris-HCl pH 7.9, 100 mM NaCl, 50% glycerol, 1 mM DTT, 0.1 mM EDTA, and 0.1% Triton X-100 through a 14 kDa cut-off dialysis tubing. The dialysis was performed at 1:100 ratio for 20 hours at 4° C. and the buffer was changed once after 4 hours. After dialysis, the sample was centrifuged at 13,000 rpm for 30 minutes and filtered through a 0.22 μm filter. The final quality control included SDS-PAGE analysis along with Western Blot. The protein concentration was determined via the Bradford method using bovine serum albumin (BSA) as a protein standard.

The other T7 RNA polymerase variants including LA and LAR were generated and purified as previously described in U.S. Pat. No. 8,105,813, the contents of which are hereby incorporated by reference in their entirety.

The mutations in each variant are listed in Table 1.

TABLE 1
T7 RNA polymerase variants

| Polymerase | |
|---|---|
| variant | Mutations |
| LA | Y639L, H784A |
| LAR | K378R, Y639L, H784A |
| LAR-M5 | K378R, Y639L, H784A, S430P, |
| | N433T, S633P, F849I, F880Y |
| LAR-M6 | K378R, Y639L, H784A, S430P, |
| | N433T, S633P, F849I, F880Y, P266L |
| RGVG-M5 | E593G, Y639V, V685A, H784G, |
| | S430P, N433T, S633P, F849I, F880Y |

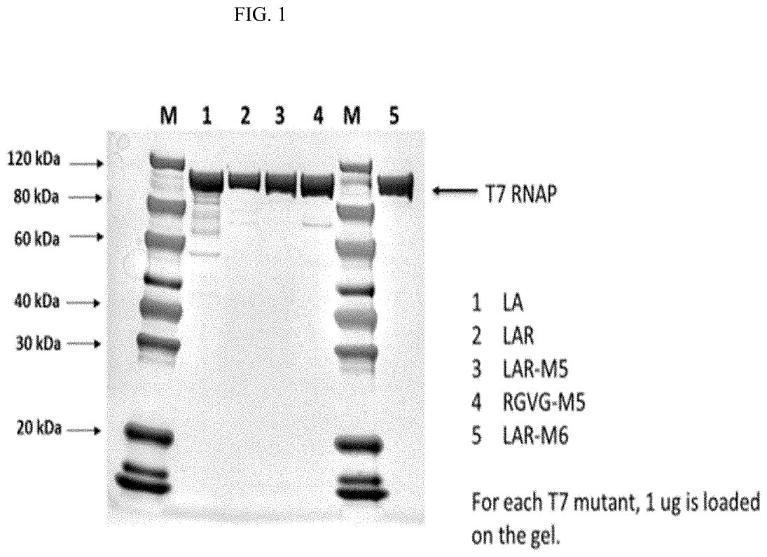

The purified T7 RNA polymerase variants were analyzed on a gradient SDS-PAGE gel (4-20%) for quality. As shown in FIG. 1, all variants exhibited high purity and corresponded to the expected molecular weight (˜99 kDa). In particular, the variants containing the LAR mutations, i.e., LAR (lane 2), LAR-M5 (lane 3) and LAR-M6 (lane 5), appeared to have better integrity compared to the other two variants, LA (lane 1) and RGVG-M5 (lane 4).

### Example 2. In Vitro Transcription Assay

In vitro transcription assay was carried out to evaluate the ability of the T7 RNA polymerase variants in incorporating 2′-O-methyl modified nucleotides. The assay was performed with two different DNA template pools, namely Pool 2 and Pool 4. The sequences of the templates and the primers used to amplify each template are shown in Table 2. In the table, “N” is any nucleotide.

TABLE 2
DNA templates and primers

| | | | SEQ ID |
|---|---|---|---|
| DNA | | Sequence (5′-3′) | NO |
| Pool 2 | DNA | GGGGAGTACAATAACGTTCTCGNNNNNNNNNNNNNNNNNNNNN | 7 |
| | template | NNNNNNNNNGGATCGTTACGACTAGCATCGATG | |
| | 5′ primer | TAATACGACTCACTATGGGGAGTACAATAACGTTCTCG | 8 |
| | 3′ primer | CATCGATGCTAGTCGTAACGATCC | 9 |
| Pool 4 | DNA | GGGAGAGCATTGCTCGTTAGTGNNNNNNNNNNNNNNNNNNNNN | 10 |
| | template | NNNNNNNNNAGCTAGTGACTCGGATCATCTAGG | |
| | 5′ primer | TAATACGACTCACTATAGGGAGAGCATTGCTCGTTAGTG | 11 |
| | 3′ primer | CCTAGATGATCCGAGTCACTAGCT | 12 |

The transcription reactions contained 200 mM HEPES/KOH pH 7.5, 6 mM Mg(OAc)₂, 2 mM spermidine, 0.5 mM each 2′-O-methyl-NTP, 40 mM DTT, 0.01% (w/v) Triton X-100, 10% (w/v) PEG-8000, 1.5 mM Mn(OAc)₂, 5 units/ml inorganic pyrophosphatase, 1 mM GMP, 100 nM DNA template, and 25 ng/μ1 T7 RNA polymerase variant. All enzymes were equalized based on mass. The reactions were incubated at 37° C. for 16 hours. After the reaction, samples were treated with 2 μl of Turbo DNase (TURBO DNA-Free™ Kit, Themo Fisher, Cat. No. AM1907) at 37° C. for 15 minutes to remove the DNA template. Transcript yields were analyzed by 10% polyacrylamide gel electrophoresis.

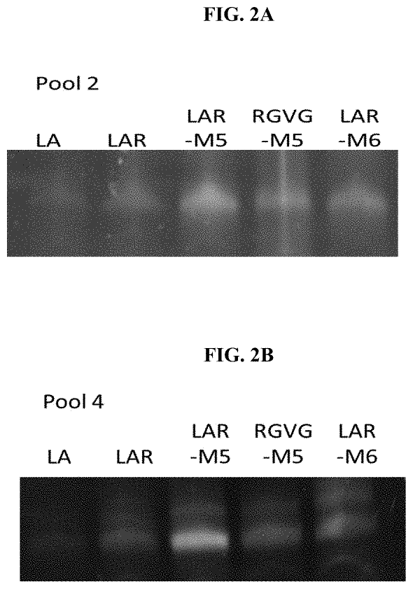

The results are shown in FIG. 2A and FIG. 2B for Pool 2 and Pool 4, respectively. Surprisingly, LAR-M5 repeatedly produced the highest yields of transcripts among the tested variants. Compared to RGVG-M5, the LAR-M5 variant produced at least 2 times more transcripts. LAR-M6 was the second best among the tested variants.

### Example 3. Sequencing Analysis for T7 RNA Polymerase Variants

Transcripts produced by each T7 RNA polymerase variant in vitro were analyzed for transcription bias. Transcription reactions with DNA template Pool 4 performed as described in Example 2 were treated with 2 μl of Turbo DNase (TURBO DNA-Free™ Kit, Themo Fisher, Cat. No. AM1907) at 37° C. for 15 minutes. The DNase was removed with the DNase inactivation reagent according to the manufacturer's protocol. 2 μl of each sample was converted to DNA via reverse transcription in a 12.5 μl reaction mix containing 2 μM 5′ primer for Pool 4 (SEQ ID NO: 11), 0.5 mM dNTPs, 1× SuperScript IV Reaction Buffer, 5 mM DTT, and 10 units SuperScript IV (Themo Fisher, Cat. No. 18090050) at 50° C. for 10 min. The samples were then amplified in a 100 μl PCR reaction containing 1× Thermo Pol buffer, 0.2 mM dNTPs, 1 μM each primer for Pool 4 (SEQ ID NOs: 11 and 12) and 5 units NEB Taq polymerase. Samples were amplified for 8 cycles (95° C. for 30 seconds, 55° C. for 30 seconds and 72° C. for 30 seconds). In order to utilize the Amplicon EZ platform at GENEWIZ (South Plainfield, N.J.), the samples needed to be 150 base pairs. Additional sequence was added via PCR to the products described above using a 5′ primer (ACACTCTTTCCCTACACGACGCTCTTCCGATCTACTGATCGAAGTACGTATGGAG CTCTCGTCTAATACGACTCACTATAGGG (SEQ ID NO: 13)) and a 3′ primer (GACTGGAGTTCAGACGTGTGCTCTTCCGATCTTAGCATGACGGATCGGTACGTC AGTATCGACCTAGATGATCCGAGTCACT (SEQ ID NO: 14)) after 7 cycles of amplification (95° C. for 30 seconds, 55° C. for 30 seconds, and 72° C. for 30 seconds), followed by 2 cycles of extension (95° C. for 30 seconds and 72° C. for 1 min). 75 μl of each PCR reaction was desalted with a Bio-Gel® P-30 spin column (Bio-Rad) and analyzed at GENEWIZ using the Amplicon EZ pipeline.

For data analysis, only samples that had a 29-31 nucleotide sequence in the random region and contained both primers sequences (SEQ ID NOs: 11 and 12) were used in the analysis. Primers were removed prior to the composition analysis. The percentage of each base was calculated based on the random region only.

The results from sequencing analysis are presented in Table 3. The composition of each base in the products produced by each T7 RNA polymerase variant was compared to the composition in the original template pool (i.e., naïve pool). It appears that while RGVG-M5 has a bias against incorporating 2′-O-methyl-ATP, all other variants including LAR-M5 do not have transcription biases.

TABLE 3
Transcription biases

| | naïve pool | LA | LAR | LAR-M5 | RGVG-M5 | LAR-M6 |
|---|---|---|---|---|---|---|
| % A | 20.9 | 20.0 | 20.1 | 20.2 | 18.6 | 20.7 |
| % T | 31.2 | 31.7 | 31.9 | 31.4 | 33.3 | 31.8 |
| % C | 24.7 | 26.4 | 26.1 | 25.5 | 25.1 | 25.0 |
| % G | 23.2 | 21.9 | 22.0 | 22.9 | 23.1 | 22.6 |
| Total number | 75,319 | 67,313 | 46,914 | 96,138 | 38,835 | 77,166 |
| of samples in | | | | | | |
| final dataset | | | | | | |

The result is important for SELEX™ application as ideally the molecular biology steps of the SELEX™ process should duplicate exactly the output pool from each round of SELEX™. Often SELEX™ experiments are carried out 5-15 rounds therefore any base bias will be amplified as further rounds of selection are carried out. Thus, the variants LAR-M5 and LAR-M6 are suitable for use in SELEX™ experiments.

### Example 4. Selection of Modified Aptamers Against Cholera Toxin Using T7RNA Polymerase Variants

A SELEX screening to identify modified aptamers that specifically bind to cholera toxin (holotoxin or B subunit only) was performed.

### Preparation of RNA Pool

A large scale of PCR reaction (50 mL) was performed following the cycle of 95° C. for 15 seconds, 60° C. for 15 seconds and 72° C. for 15 seconds. The PCT template includes a synthesized library of oligonucleotides, each containing a random sequence of 30 nucleotides in length. The PCR products were precipitated and purified away from any unincorporated substrates. The purified DNA library (the PCR products) was resuspended in TE buffer for late use. A library of unique transcription templates was generated by this PCR amplification of the synthetic DNAs.

To generate a library of 2′-OMe modified oligonucleotides, a large scale of in vitro transcription reaction was performed using T7 polymerase variants and 2′-OMe NTPs (A, T, G and U) as substrates. A 10 ml reaction using the double stranded PCR products as transcription templates was performed. The experiments and optimal concentrations of metal ions (e.g., manganese) described herein were used with a mNTP mixture in which each of the four mNTPs was at a concentration of 0.5 mM. The transcription mixture was prepared as shown in Table 4.

TABLE 4
Transcription using mutant T7 enzyme

| Components | Final Concentration | |
|---|---|---|
| water | Bringing volume | |
| | up to 10 mL | |
| 5X TC buffer | 1X TC | |
| 1000 mM Mg(OAC)2 | 15 | mM |
| 20 mM Mn(OAC)2 | 1.5 | mM |
| 100 mM mATP | 0.5 | mM |
| 100 mM mCTP | 0.5 | mM |
| 100 mM mGTP | 0.5 | mM |
| 100 mM mUTP | 0.5 | mM |
| 50% PEG-8000 | 10% | |
| units/ml Ppi (NEB) | 5 | U/ml |
| 10 mM GMP | 1 | mM |
| Mutant T7 (500 ng/ul) | 25 | ng/ul |
| DNA Template | 200 | nM |

The mixture was incubated overnight at 37° C. After gel purification (10% denaturing polyacrylamide gel) and DNase treatment, the modified transcripts were precipitated, resuspended and aliquoted in water for filter and plate SELEX selections.

### Filter SELEX Screening

The protocol of filter based SELEX screening follows the steps of:
1. Mixing cholera toxin (target) and the aliquoted RNA in a total volume of 100 μl PBS with calcium, magnesium (DPBS+ buffer);
2. Incubating the binding reaction for 1 hour at 37° C.;
3. Pretreating the Nitrocellulose filter (Centrex Centrifuge Filters 5 mL, 0.45 μm), including incubating the column with 1 ml 0.5M KOH for 15 min at room temperature, spinning the pretreated column, and washing the column one time with 1 ml water and one time with 1 ml DPBS (+) buffer;
4. Loading the binding reaction onto the KOH pretreated column and spinning the column at 1500 rpm for 1 min and discarding the flow-through;
5. Washing the column twice with 500 W DPBS and eluting the column with 200 μl elution buffer (7M Urea, 300 mM Sodium Acetate, 3 mM EDTA) heated to 90° C.; spinning the column after 1 min incubation and collecting the flow-through;
6. Repeating the elution and combining the collected samples;
7. Precipitating the collected RNA;
8. Reversely transcribing the collected RNA using superscript IV protocol (100 μl total reaction);
9. Amplifying the entire reversely transcribed products (cDNA) with PCR in 400 μl PCR reaction;
10. Purifying the PCR products and precipitating 100 μl of the PCR products and using the precipitant as template in 200 μl transcription reaction for overnight at 37° C.; and
11. Purifying the transcripts and the generated modified transcripts are used for input into the round of selection, either for another round of filter based SELEX screening or for plate SELEX selection processes.

### Plate SELEX Screening

The protocol for plate-based SELEX screening follows the steps of:
1. Incubating 20 pmoles choleta toxin in 10 μl DPBS+ buffer in the wells of a 96-well plate (Nunc MaxiSorp™), at 37° C. for 1 hour and washing the plate twice with 200 μl DPBS+ buffer; the target proteins immobilized by hydrophobic absorption to the surface of the 96-well plate.
2. Blocking the target-immobilized wells with 100 μl DPBS+ buffer containing 0.1 mg/ml BSA at 37° C. for 1 hour; washing the blocked plate three times with 200 μl DBPS+ buffer;
3. Adding to each well the transcripts collected from the filter SELEX selection (100 μl per well);
4. Incubating the plate at 37° C. for 1 hour; discarding the supernatants and washing the plate three times with 200 μl DBPS+ buffer;
5. Reversely transcribing the products (bound sequences) in the plate (in situ reverse transcription) using superscript IV protocol (100 μl total reaction);
6. Amplifying the entire reverse transcribed cDNA products with PCR in 400 μl PCR reaction;
7. Precipitating 100 μl of the PCR products and using the precipitant as template of the transcription reaction (200 μl reaction) at 37° C. overnight; and
8. Purifying the transcripts for the subsequent selection step. The PCR amplification follows the same PCR conditions as used in the large scale preparation of the template DNAs.
After more than 5 rounds of selection and amplification, the binding characteristics of the selected sequences to CT holotoxin and/or CT B subunit was assessed by a plate binding assay.

### Target Binding Assay

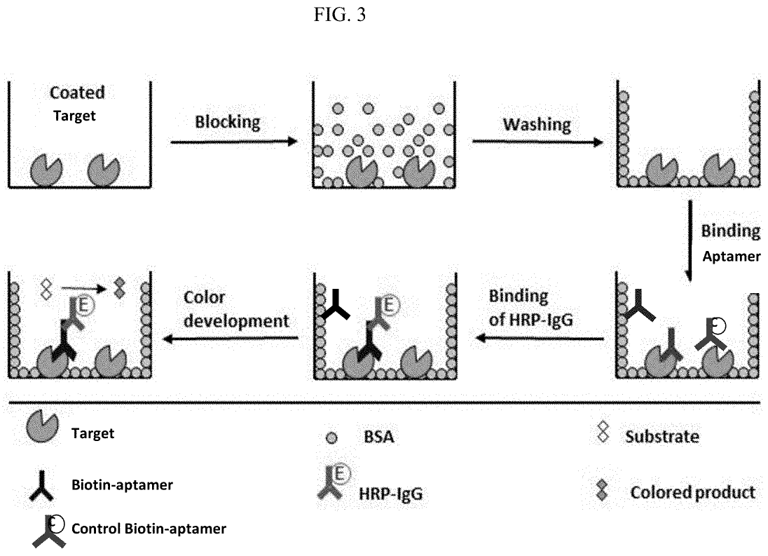

The sequence candidates from the filter and plate SELEX selections were assessed for target protein binding. FIG. 3 shows a diagram illustrating the plate-based ELISA binding assay to validate the interaction of the selected clones to their target proteins. The target protein (cholera holotoxin and B subunit) binding assay includes the steps of:
1. Diluting the selected sequence clones to a concentration of 10 μM each;
2. Immobilizing cholera holotoxin or cholera toxin B subunit on a 96-well plate (Nunc MaxiSorp™) at a concentration of 0.5 μg/well by incubating the plate at 4° C. overnight (alternatively 1 hour at room temperature) and washing the plate three times with 200 μl DBPS+ buffer (1×);
3. Blocking the plate with 200 μl DPBS+ buffer (1×) containing 25 mg/mL BSA at room temperature for 1 hour; washing the blocked plate three times with 200 μl DBPS+ buffer (1×);
4. Titrating the clones and tRNAs to 100 nM in 100 μl DBPS+ buffer (2×);
5. Adding the titrates to the plate and incubating the mix at room temperature for 30 minutes with shaking; washing the plates three times with 200 μl DBPS+ buffer (2×);
6. Adding 100 μl substrate (1-Step Ultra TMB-ELISA, (Pierce, #34028)) for 5 minutes at room temperature;
7. Adding 100 μl 2N H₂SO₄ to stop the reaction; and
8. Reading the reaction at 450 nm.

Two phases of SELEX screening processes were carried for oligonucleotide binding to cholera holotoxin and/or CT B subunit. The filter-based selections and plate-based selections were repeated for several rounds. In each selection round, the RNA and DNA pools were sequenced and the modifications were tested.

Unique sequences that specifically bind to cholera holotoxin and/or B subunit were identified and sequenced (Table 5).

TABLE 5
Aptamers specific to Cholera toxin (holotoxin and B subunit)

| | Sequence (5′-3′) | SEQ | Sequence (5′-3′) | SEQ |
|---|---|---|---|---|
| Clone | (central unique | ID | (full sequence including the | ID |
| NO. | sequence region) | NO. | constant 5′ end and 3′ end regions) | NO. |
| 1 | TCGTCTCGGAGTTTAT | 15 | GGGAGAGCATTGCTCGTTAGTGTCGTC | 16 |
| | CTCCGTGGTCGTTGA | | TCGGAGTTTATCTCCGTGGTCGTTGAA | |
| | | | GCTAGTGACTCGGATCATCTAGG | |
| 2 | TCGTCTTGGTAATTCC | 17 | GGGAGAGCATTGCTCGTTAGTGTCGT | 18 |
| | CTTTCTGGTCGTTTA | | CTTGGTAATTCCCTTTCTGGTCGTTTA | |
| | | | AGCTAGTGACTCGGATCATCTAGG | |
| 3 | TCGTCTTAAACTTCGT | 19 | GGGAGAGCATTGCTCGTTAGTGTCGTC | 20 |
| | GTTTTTGGTCGTTGT | | TTAAACTTCGTGTTTTTGGTCGTTGTA | |
| | | | GCTAGTGACTCGGATCATCTAGG | |
| 4 | TCGTCTTCAATCATGC | 21 | GGGAGAGCATTGCTCGTTAGTGTCGTC | 22 |
| | ATTGTTGGTCGTTGA | | TTCAATCATGCATTGTTGGTCGTTGAA | |
| | | | GCTAGTGACTCGGATCATCTAGG | |
| 5 | TCGTCTCGCGAACGTT | 23 | GGGAGAGCATTGCTCGTTAGTGTCGT | 24 |
| | AATCGCGTGGTCGTG | | CTCGCGAACGTTAATCGCGTGGTCGT | |
| | | | GAGCTAGTGACTCGGATCATCTAGG | |
| 6 | TCGTCTCGAATCGAAC | 25 | GGGAGAGCATTGCTCGTTAGTGTCGTC | 26 |
| | TTTCGTGGTCGTTAA | | TCGAATCGAACTTTCGTGGTCGTTAAA | |
| | | | GCTAGTGACTCGGATCATCTAGG | |
| 7 | TCGTCTTGACCTTTGC | 27 | GGGAGAGCATTGCTCGTTAGTGTCGTC | 28 |
| | TGGTCATGGTCGTAA | | TTGACCTTTGCTGGTCATGGTCGTAAA | |
| | | | GCTAGTGACTCGGATCATCTAGG | |
| 8 | TCGTCTAATCGGTTTT | 29 | GGGAGAGCATTGCTCGTTAGTGTCGTC | 30 |
| | CCGGTTTGGTCGTTG | | TAATCGGTTTTCCGGTTTGGTCGTTGA | |
| | | | GCTAGTGACTCGGATCATCTAGG | |
| 9 | TCGTCTCGGGATTTTT | 31 | GGGAGAGCATTGCTCGTTAGTGTCGTC | 32 |
| | CCCGTGGTCGTTCAT | | TCGGGATTTTTCCCGTGGTCGTTCATA | |
| | | | GCTAGTGACTCGGATCATCTAGG | |
| 10 | TCGTCTCGAGTTGTGA | 33 | GGGAGAGCATTGCTCGTTAGTGTCGTC | 34 |
| | TTACTCGTGGTCGTA | | TCGAGTTGTGATTACTCGTGGTCGTAA | |
| | | | GCTAGTGACTCGGATCATCTAGG | |
| 11 | TCGTCTCATCGAATGG | 35 | GGGAGAGCATTGCTCGTTAGTGTCGTC | 36 |
| | TCGGTGTGGTCGTTA | | TCATCGAATGGTCGGTGTGGTCGTTA | |
| | | | AGCTAGTGACTCGGATCATCTAGG | |

In addition to the constant sequences at the 5′ end (5′ GGGAGAGCATTGCTCGTTAGTG 3′; SEQ ID NO.: 37) and 3′-end (5′AGCTAGTGACTCGGATCATCTAGG 3′; SEQ ID NO.: 38) of each aptamer, sequence alignment of these aptamer candidates indicate that they share 6 conserved nucleotides 5′ TCGTCT3′ (SEQ ID NO.: 39) and 7 conserved nucleotides 5′ TGGTCGT3′ (SEQ ID NO.: 40).

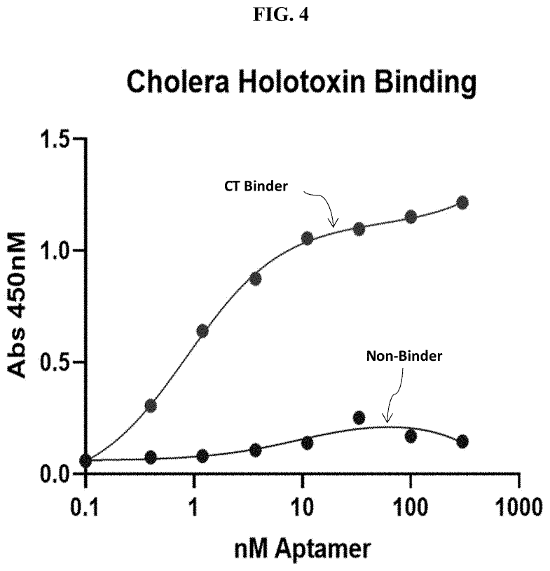

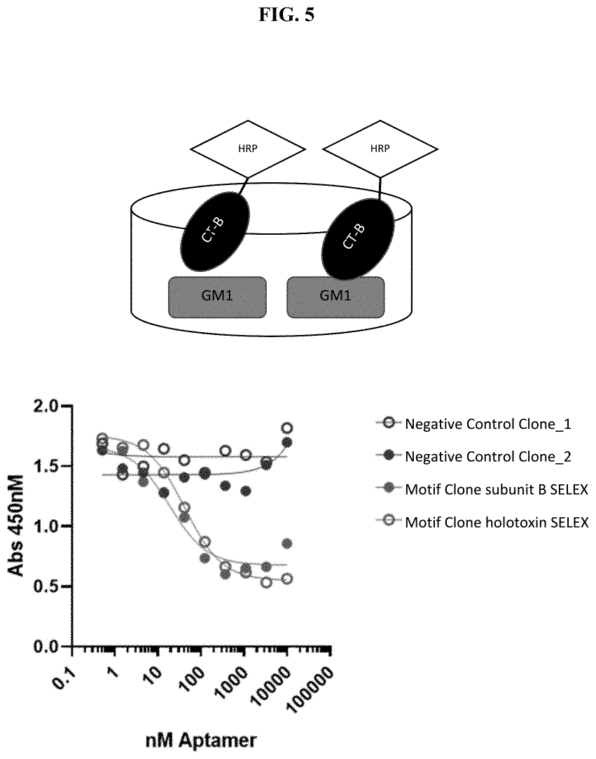

These candidates all demonstrate a specific binding to cholera toxin (FIG. 4). These aptamer candidates demonstrate an active function in the competition assay (as shown in FIG. 5). In a competition assay, a plate was coated with 2 μg/ml GM1 and blocked with recombinant BSA. 6 nM HRP-labeled cholera toxin (CT) B subunit was combined with unlabeled CT B subunit. A titration of candidate aptamers selected from the screening were validated for the binding to CT B subunit.

### Example 5. Selection of Modified Aptamers Against 4-1BB Using T7 RNA Polymerase Variants

A SELEX screening for aptamers against 4-1BB (also known as CD137) was performed. The screening process follows the procedures discussed in Example 4.

TABLE 6
Aptamers specific to 4-1BB

| | | SEQ |
|---|---|---|
| | Sequence (5′-3′) | ID |
| Clone | (Central region sequence) | NO. |
| W2.S3.1_R6F_21 | AACGAUAUGGUCCCGGAAGUUGGGCCUCGUU | 41 |
| W2.S3.1_R6F_22 | GCAUGAGUGACCAUAUAGGUUGCCGCGCUCG | 42 |
| W2.S3.1_R6F_6 | GACUGCGGUGCGUUGAUGCCGUGUGUUGCUC | 43 |

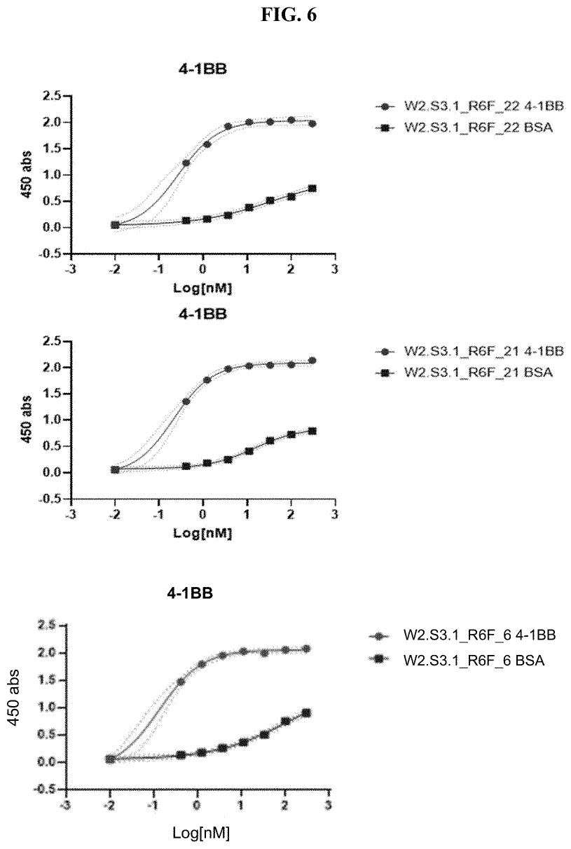

In the binding assay, three aptamer candidates (Table 6) indicate specific binding to 4-1BB as shown in FIG. 6.

### Example 6. Selection of Modified Aptamers Against KRAS Using T7 RNA Polymerase Variants

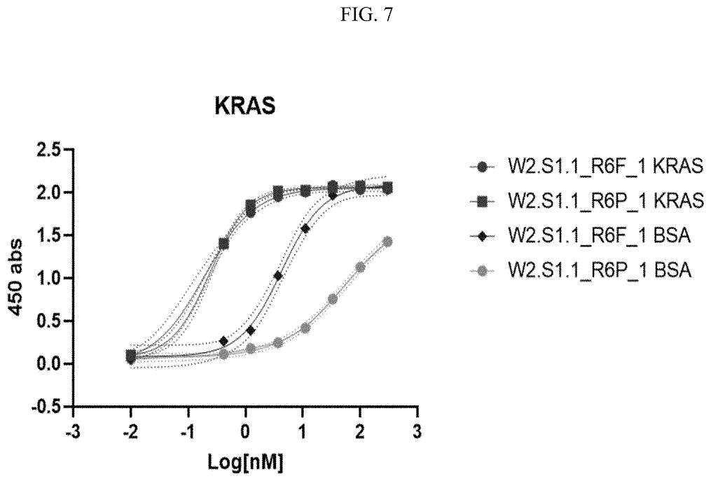

A SELEX screening for aptamers against KRAS was performed. The screening process follows the procedures discussed in Example 4. FIG. 7 illustrates the binding assay of two positive clones (Table 7) to KRAS, as compared to the control material BSA.

TABLE 7
Aptamers specific to KRAS

| | Sequence (5′-3′) | SEQ ID |
|---|---|---|
| Clone | (Central region sequence) | NO. |
| W2.S1.1_R6F-1 | GCGUUACAGCAGUUGCCACAAGGCACGUUUU | 44 |
| W2.S1.1_R6P-1 | GAGCCUAUGGUUGUUGUGUGCUCCUCGCGGG | 45 |

### EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

This listing replaces all prior versions and listings of the claims:

1. A T7 RNA polymerase variant, comprising one or more mutations relative to the wild-type polypeptide sequence set forth in SEQ ID NO.: 1, wherein the one or more mutations comprise:
a. at least one amino acid substitution selected from the group consisting of K378R, Y639L and H784A; and
b. at least one amino acid substitution selected from the group of consisting of S430P, N433T, S633P, F849I, F880Y and P266L.

2. The T7 RNA polymerase variant of claim 1, wherein the one or more mutations comprise:
a. one of the following sets of amino acid substitutions:
i. Y639L and H784A; or
ii. K378R, Y639L and H784A; and
b. one of the following sets of amino acid substitutions:
i. S430P, N433T, S633P, F849I and F880Y; or
ii. S430P, N433T, S633P, F849I, F880Y and P266L.

3. The T7 RNA polymerase variant of claim 2, wherein the one or more mutations comprise:
a. K378R, Y639L and H784A; and
b. S430P, N433T, S633P, F849I and F880Y.

4. The T7 RNA polymerase variant of claim 2, wherein the one or more mutations comprise:
a. K378R, Y639L and H784A; and
b. S430P, N433T, S633P, F849I, F880Y and P266L.

5. The T7 RNA polymerase variant of claim 1 further comprising a protein tag, wherein the protein tag is attached to the N-terminus of the polymerase variant, or the C-terminus of the polymerase variant.

6. (canceled)

7. The T7 RNA polymerase variant of claim 5, wherein the protein tag is a polyhistidine-tag.

8. A T7 RNA polymerase variant, comprising a polypeptide sequence selected from the group consisting of the sequences set forth in SEQ ID NO.: 2 and 5, or a functional fragment thereof.

9. (canceled)

10. The T7 RNA polymerase variant of claim 1, wherein said T7 RNA polymerase variant has enhanced ability to incorporate a 2′ modified nucleotide compared to the wild-type T7 RNA polymerase.

11. The T7 RNA polymerase variant of claim 10, wherein said 2′ modified nucleotide is selected from the group consisting of 2′-O-methyl ATP, 2′-O-methyl CTP, 2′-O-methyl UTP, 2′-O-methyl GTP, 2′-fluoro ATP, 2′-fluoro CTP, 2′-fluoro UTP, 2′-fluoro GTP, 2′-amino ATP, 2′-amino CTP, 2′-amino UTP, and 2′-amino GTP.

12. The T7 RNA polymerase variant of claim 11, wherein said 2′ modified nucleotide is 2′-O-methyl ATP, 2′-O-methyl CTP, 2′-O-methyl UTP, or 2′-O-methyl GTP.

13. The T7 RNA polymerase variant of claim 10, wherein said T7 RNA polymerase variant does not have a bias in incorporating 2′-O-methyl ATP, 2′-O-methyl CTP, 2′-O-methyl UTP, or 2′-O-methyl GTP.

14. The T7 RNA polymerase variant of claim 1, wherein said T7 RNA polymerase variant has enhanced thermostability compared to the wild-type T7 RNA polymerase.

15. A nucleic acid molecule comprising a nucleotide sequence encoding a T7 RNA polymerase variant, wherein the nucleotide sequence comprises a polynucleotide sequence set forth in SEQ ID NO.: 3, or a polynucleotide sequence set forth in SEQ ID NO.: 6.

16. The nucleic acid molecule of claim 15, wherein the sequence of the nucleic acid molecule is codon optimized for expression in a protein expression system.

17. The nucleic acid molecule of claim 16, wherein the protein expression system is an E. coli expression system.

18. (canceled)

19. (canceled)

20. (canceled)

21. (canceled)

22. (canceled)

23. A method of synthesizing a 2′ modified polynucleotide, comprising contacting a template nucleic acid with a T7 RNA polymerase variant in the presence of 2′ modified nucleoside triphosphates under conditions that allow synthesis of the 2′-modified polynucleotide by the polymerase activity of the T7 RNA polymerase variant,
wherein the T7 RNA polymerase variant comprises one or more mutations relative to the wild-type polypeptide sequence set forth in SEQ ID NO.: 1, wherein the one or more mutations comprise:
a. at least one amino acid substitution selected from the group consisting of K378R, Y639L and H784A; and;
b. at least one amino acid substitution selected from the group of consisting of S430P, N433T, S633P, F849I, F880Y and P266L.

24. The method of claim 23, wherein the 2′ modified nucleoside triphosphates are one or more nucleoside triphosphates selected from the group consisting of 2′-O-methyl ATP, 2′-O-methyl CTP, 2′-O-methyl UTP, 2′-O-methyl GTP, 2′-fluoro ATP, 2′-fluoro CTP, 2′-fluoro UTP, 2′-fluoro GTP, 2′-amino ATP, 2′-amino CTP, 2′-amino UTP, and 2′-amino GTP.

25. The method of claim 24, wherein the 2′ modified nucleoside triphosphates are one or more nucleoside triphosphates selected from the group consisting of 2′-O-methyl ATP, 2′-O-methyl CTP, 2′-O-methyl UTP, and 2′-O-methyl GTP.

26. (canceled)

27. (canceled)

28. The method of claim 25, wherein the 2′ modified polynucleotide is a resistant to nucleases and/or base hydrolysis.

29. A modified nucleic acid molecule comprising one or more 2′ modified nucleotide units, wherein the modified nucleic acid molecule is synthesized by a method that comprises:
contacting a template nucleic acid with a T7 RNA polymerase variant in the presence of 2′ modified nucleoside triphosphates under conditions that allow synthesis of the 2′-modified nucleic acid molecule by the polymerase activity of the T7 RNA polymerase variant,
wherein the T7 RNA polymerase variant comprises one or more mutations relative to the Attorney Docket No.: 2125.1000US371 wild-type polypeptide sequence set forth in SEQ ID NO.: 1, wherein the one or more mutations comprise:
a. at least one amino acid substitution selected from the group consisting of K378R, Y639L and H784A; and;
b. at least one amino acid substitution selected from the group of consisting of S430P, N433T, S633P, F849I, F880Y and P266L.

30. The modified nucleic acid molecule of claim 29, wherein the modified nucleic acid molecule is a nucleic acid aptamer that binds to a target of interest with a high specificity and affinity.

31. (canceled)

32. The modified nucleic acid molecule of claim 30, wherein the target of interest is a protein, a polypeptide, a peptide, a nucleic acid, a polysaccharide, a lipid molecule, or a chemical compound.

33. The modified nucleic acid molecule of claim 32 wherein the target of interest is a protein.


SEQUENCE LISTING (45 sequences)

SEQ ID NO: 1 - protein, 883 aa, Bacteriophage T7
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 2 - protein, 883 aa, Artificial sequence
  Description of Artificial Sequence Synthetic polypeptide
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWRRAAAAVYRKDKARKSRRISLEFMLEQANKFANHKAIWFPYNMDWRGRVYAVPMFTPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRPVMTLALGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVASQDGSHLRKTVVWAHEKYGIESFALIHDSFGTIPADAANLFKAVRETMVDTYESCDVLADFYDQIADQLHESQLDKMPALPAKGNLNLRDILESDYAFA

SEQ ID NO: 3 - DNA, 2652 bp, Artificial Sequence
  Description of Artificial Sequence Synthetic polynucleotide
atgaacacaataaacatagctaagaacgactttagcgacatcgaactggcggcgattccgtttaacaccctggcggaccactatggcgagcgtctggcgcgtgaacagctggcgctggagcacgaaagctatgagatgggtgaagcgcgtttccgtaagatgtttgagcgtcaactgaaagcgggcgaagttgcggacaacgcggcggcgaagccgctgattaccaccctgctgccgaaaatgatcgcgcgtattaacgattggttcgaggaagttaaggcgaaacgtggcaagcgtccgaccgcgttccagtttctgcaagagatcaagccggaagcggtggcgtacatcaccattaaaaccaccctggcgtgcctgaccagcgcggacaacaccaccgttcaagcggttgcgagcgcgattggtcgtgcgattgaggacgaagcgcgttttggccgtattcgtgatctggaggcgaagcacttcaagaaaaacgttgaggaacagctgaacaaacgtgtgggtcacgtttataagaaagcgtttatgcaagtggttgaagcggacatgctgagcaaaggcctgctgggtggcgaggcgtggagcagctggcacaaagaagatagcatccacgtgggtgttcgttgcatcgagatgctgattgaaagcaccggcatggtgagcctgcaccgtcagaacgcgggcgtggttggtcaagacagcgaaaccatcgaactggcgccggagtacgcggaagcgatcgcgacccgtgcgggcgcgctggcgggtatcagcccgatgttccagccgtgcgtggttccgccgaaaccgtggaccggtattaccggtggcggttactgggcgaacggtcgtcgtccgctggcgctggtgcgtacccacagcaagaaagcgctgatgcgttacgaggatgtttatatgccggaagtgtataaggcgatcaacattgcgcaaaacaccgcgtggaaaattaacaagaaagtgctggcggttgcgaacgtgatcaccaagtggaaacactgcccggttgaggacatcccggcgattgaacgtgaggaactgccgatgaagccggaggacatcgatatgaacccggaagcgctgaccgcgtggcgtcgtgcggcggcggcggtgtaccgtaaggataaagcgcgcaaaagccgtcgtattagcctggagttcatgctggaacaggcgaacaagtttgcgaaccacaaagcgatctggttcccgtacaacatggactggcgtggtcgtgtttatgcggtgccaatgttcaccccgcaaggcaacgatatgaccaagggtctgctgaccctggcgaagggcaaaccgattggcaaagagggttactattggctgaaaatccacggcgcgaactgcgcgggtgttgacaaggtgccgttcccggagcgtatcaagttcatcgaggaaaaccacgaaaacatcatggcgtgcgcgaaaagcccgctggagaacacctggtgggcggaacaggatagcccgttctgctttctggcgttctgctttgaatacgcgggcgttcaacaccacggtctgagctataactgcagcctgccgctggcgtttgatggcagctgcagcggtatccagcacttcagcgcgatgctgcgtgatgaggttggcggtcgcgcggtgaacctgctgccgagcgaaaccgtgcaggacatctacggtattgttgcgaagaaagtgaacgagatcctgcaagcggacgcgattaacggtaccgataacgaggtggttaccgttaccgatgaaaacaccggcgagatcagcgaaaaggtgaaactgggtaccaaggcgctggcgggccagtggctggcgtatggcgttacccgtagcgtgaccaagcgtccagttatgaccctggcgctgggcagcaaagagttcggttttcgtcagcaagtgctggaagacaccatccaaccggcgattgatagcggcaagggtctgatgtttacccagccgaaccaagcggcgggttacatggcgaaactgatctgggagagcgtgagcgttaccgtggttgcggcggttgaagcgatgaactggctgaagagcgcggcgaaactgctggcggcggaagtgaaggacaagaaaaccggtgaaattctgcgtaaacgttgcgcggttcactgggtgaccccggatggcttcccggtttggcaggagtataagaaaccgatccaaacccgtctgaacctgatgttcctgggccagtttcgtctgcaaccgaccatcaacaccaacaaggacagcgagattgatgcgcacaaacaggaaagcggtattgcgccgaactttgtggcgagccaagacggcagccacctgcgtaagaccgtggtttgggcgcacgagaaatacggtatcgagagcttcgcgctgattcacgacagctttggtaccatcccggcggatgcggcgaacctgttcaaggcggttcgtgaaaccatggtggacacctacgaaagctgcgatgttctggcggacttctatgatcagatcgcggaccaactgcacgaaagccagctggataaaatgccggcgctgccggcgaagggtaacctgaacctgcgtgacattctggagagcgattatgcgtttgcgtaa

SEQ ID NO: 4 - protein, 9 aa, Artificial Sequence
  Description of Artificial Sequence Synthetic polypeptide
MHHHHHHGS

SEQ ID NO: 5 - protein, 892 aa, Artificial Sequence
  Description of Artificial Sequence Synthetic polypeptide
MHHHHHHGSMNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWRRAAAAVYRKDKARKSRRISLEFMLEQANKFANHKAIWFPYNMDWRGRVYAVPMFTPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRPVMTLALGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVASQDGSHLRKTVVWAHEKYGIESFALIHDSFGTIPADAANLFKAVRETMVDTYESCDVLADFYDQIADQLHESQLDKMPALPAKGNLNLRDILESDYAFA

SEQ ID NO: 6 - DNA, 2679 bp, Artificial Sequence
  Description of Artificial Sequence Synthetic polynucleotide
atgcatcaccaccaccaccacggatcaatgaacacaataaacatagctaagaacgactttagcgacatcgaactggcggcgattccgtttaacaccctggcggaccactatggcgagcgtctggcgcgtgaacagctggcgctggagcacgaaagctatgagatgggtgaagcgcgtttccgtaagatgtttgagcgtcaactgaaagcgggcgaagttgcggacaacgcggcggcgaagccgctgattaccaccctgctgccgaaaatgatcgcgcgtattaacgattggttcgaggaagttaaggcgaaacgtggcaagcgtccgaccgcgttccagtttctgcaagagatcaagccggaagcggtggcgtacatcaccattaaaaccaccctggcgtgcctgaccagcgcggacaacaccaccgttcaagcggttgcgagcgcgattggtcgtgcgattgaggacgaagcgcgttttggccgtattcgtgatctggaggcgaagcacttcaagaaaaacgttgaggaacagctgaacaaacgtgtgggtcacgtttataagaaagcgtttatgcaagtggttgaagcggacatgctgagcaaaggcctgctgggtggcgaggcgtggagcagctggcacaaagaagatagcatccacgtgggtgttcgttgcatcgagatgctgattgaaagcaccggcatggtgagcctgcaccgtcagaacgcgggcgtggttggtcaagacagcgaaaccatcgaactggcgccggagtacgcggaagcgatcgcgacccgtgcgggcgcgctggcgggtatcagcccgatgttccagccgtgcgtggttccgccgaaaccgtggaccggtattaccggtggcggttactgggcgaacggtcgtcgtccgctggcgctggtgcgtacccacagcaagaaagcgctgatgcgttacgaggatgtttatatgccggaagtgtataaggcgatcaacattgcgcaaaacaccgcgtggaaaattaacaagaaagtgctggcggttgcgaacgtgatcaccaagtggaaacactgcccggttgaggacatcccggcgattgaacgtgaggaactgccgatgaagccggaggacatcgatatgaacccggaagcgctgaccgcgtggcgtcgtgcggcggcggcggtgtaccgtaaggataaagcgcgcaaaagccgtcgtattagcctggagttcatgctggaacaggcgaacaagtttgcgaaccacaaagcgatctggttcccgtacaacatggactggcgtggtcgtgtttatgcggtgccaatgttcaccccgcaaggcaacgatatgaccaagggtctgctgaccctggcgaagggcaaaccgattggcaaagagggttactattggctgaaaatccacggcgcgaactgcgcgggtgttgacaaggtgccgttcccggagcgtatcaagttcatcgaggaaaaccacgaaaacatcatggcgtgcgcgaaaagcccgctggagaacacctggtgggcggaacaggatagcccgttctgctttctggcgttctgctttgaatacgcgggcgttcaacaccacggtctgagctataactgcagcctgccgctggcgtttgatggcagctgcagcggtatccagcacttcagcgcgatgctgcgtgatgaggttggcggtcgcgcggtgaacctgctgccgagcgaaaccgtgcaggacatctacggtattgttgcgaagaaagtgaacgagatcctgcaagcggacgcgattaacggtaccgataacgaggtggttaccgttaccgatgaaaacaccggcgagatcagcgaaaaggtgaaactgggtaccaaggcgctggcgggccagtggctggcgtatggcgttacccgtagcgtgaccaagcgtccagttatgaccctggcgctgggcagcaaagagttcggttttcgtcagcaagtgctggaagacaccatccaaccggcgattgatagcggcaagggtctgatgtttacccagccgaaccaagcggcgggttacatggcgaaactgatctgggagagcgtgagcgttaccgtggttgcggcggttgaagcgatgaactggctgaagagcgcggcgaaactgctggcggcggaagtgaaggacaagaaaaccggtgaaattctgcgtaaacgttgcgcggttcactgggtgaccccggatggcttcccggtttggcaggagtataagaaaccgatccaaacccgtctgaacctgatgttcctgggccagtttcgtctgcaaccgaccatcaacaccaacaaggacagcgagattgatgcgcacaaacaggaaagcggtattgcgccgaactttgtggcgagccaagacggcagccacctgcgtaagaccgtggtttgggcgcacgagaaatacggtatcgagagcttcgcgctgattcacgacagctttggtaccatcccggcggatgcggcgaacctgttcaaggcggttcgtgaaaccatggtggacacctacgaaagctgcgatgttctggcggacttctatgatcagatcgcggaccaactgcacgaaagccagctggataaaatgccggcgctgccggcgaagggtaacctgaacctgcgtgacattctggagagcgattatgcgtttgcgtaa

SEQ ID NO: 7 - DNA, 76 bp, Artificial Sequence
  Description of Artificial Sequence Synthetic polynucleotide
  misc_feature (23)..(52): n is a, c, t, or g
ggggagtacaataacgttctcgnnnnnnnnnnnnnnnnnnnnnnnnnnnnnnggatcgttacgactagcatcgatg

SEQ ID NO: 8 - DNA, 38 bp, Artificial Sequence
  Description of Artificial Sequence Synthetic polynucleotide
taatacgactcactatggggagtacaataacgttctcg

SEQ ID NO: 9 - DNA, 24 bp, Artificial Sequence
  Description of Artificial Sequence Synthetic polynucleotide
catcgatgctagtcgtaacgatcc

SEQ ID NO: 10 - DNA, 76 bp, Artificial Sequence
  Description of Artificial Sequence Synthetic polynucleotide
  misc_feature (23)..(52): n is a, c, t, or g
gggagagcattgctcgttagtgnnnnnnnnnnnnnnnnnnnnnnnnnnnnnnagctagtgactcggatcatctagg

SEQ ID NO: 11 - DNA, 39 bp, Artificial Sequence
  Description of Artificial Sequence Synthetic polynucleotide
taatacgactcactatagggagagcattgctcgttagtg

SEQ ID NO: 12 - DNA, 24 bp, Artificial Sequence
  Description of Artificial Sequence Synthetic polynucleotide
cctagatgatccgagtcactagct

SEQ ID NO: 13 - DNA, 83 bp, Artificial Sequence
  Description of Artificial Sequence Synthetic polynucleotide
acactctttccctacacgacgctcttccgatctactgatcgaagtacgtatggagctctcgtctaatacgactcactataggg

SEQ ID NO: 14 - DNA, 82 bp, Artificial Sequence
  Description of Artificial Sequence Synthetic polynucleotide
gactggagttcagacgtgtgctcttccgatcttagcatgacggatcggtacgtcagtatcgacctagatgatccgagtcact

SEQ ID NO: 15 - DNA, 31 bp, Artificial Sequence
  Description of Artificial Sequence Synthetic polynucleotide
tcgtctcggagtttatctccgtggtcgttga

SEQ ID NO: 16 - DNA, 77 bp, Artificial Sequence
  Description of Artificial Sequence Synthetic polynucleotide
gggagagcattgctcgttagtgtcgtctcggagtttatctccgtggtcgttgaagctagtgactcggatcatctagg

SEQ ID NO: 17 - DNA, 31 bp, Artificial Sequence
  Description of Artificial Sequence Synthetic polynucleotide
tcgtcttggtaattccctttctggtcgttta

SEQ ID NO: 18 - DNA, 77 bp, Artificial Sequence
  Description of Artificial Sequence Synthetic polynucleotide
gggagagcattgctcgttagtgtcgtcttggtaattccctttctggtcgtttaagctagtgactcggatcatctagg

SEQ ID NO: 19 - DNA, 31 bp, Artificial Sequencce
  Description of Artificial Sequence Synthetic polynucleotide
tcgtcttaaacttcgtgtttttggtcgttgt

SEQ ID NO: 20 - DNA, 77 bp, Artificial Sequence
  Description of Artificial Sequence Synthetic polynucleotide
gggagagcattgctcgttagtgtcgtcttaaacttcgtgtttttggtcgttgtagctagtgactcggatcatctagg

SEQ ID NO: 21 - DNA, 31 bp, Artificial Sequence
  Description of Artificial Sequence Synthetic polynucleotide
tcgtcttcaatcatgcattgttggtcgttga

SEQ ID NO: 22 - DNA, 77 bp, Artificial Sequence
  Description of Artificial Sequence Synthetic polynucleotide
gggagagcattgctcgttagtgtcgtcttcaatcatgcattgttggtcgttgaagctagtgactcggatcatctagg

SEQ ID NO: 23 - DNA, 31 bp, Artificial Sequence
  Description of Artificial Sequence Synthetic polynucleotide
tcgtctcgcgaacgttaatcgcgtggtcgtg

SEQ ID NO: 24 - DNA, 77 bp, Artificial Sequence
  Description of Artificial Sequence Synthetic polynucleotide
gggagagcattgctcgttagtgtcgtctcgcgaacgttaatcgcgtggtcgtgagctagtgactcggatcatctagg

SEQ ID NO: 25 - DNA, 31 bp, Artificial Sequence
  Description of Artificial Sequence Synthetic polynucleotide
tcgtctcgaatcgaactttcgtggtcgttaa

SEQ ID NO: 26 - DNA, 77 bp, Artificial Sequence
  Description of Artificial Sequence Synthetic polynucleotide
gggagagcattgctcgttagtgtcgtctcgaatcgaactttcgtggtcgttaaagctagtgactcggatcatctagg

SEQ ID NO: 27 - DNA, 31 bp, Artificial Sequence
  Description of Artificial Sequence Synthetic polynucleotide
tcgtcttgacctttgctggtcatggtcgtaa

SEQ ID NO: 28 - DNA, 77 bp, Artificial Sequence
  Description of Artificial Sequence Synthetic polynucleotide
gggagagcattgctcgttagtgtcgtcttgacctttgctggtcatggtcgtaaagctagtgactcggatcatctagg

SEQ ID NO: 29 - DNA, 31 bp, Artificial Sequence
  Description of Artificial Sequence Synthetic polynucleotide
tcgtctaatcggttttccggtttggtcgttg

SEQ ID NO: 30 - DNA, 77 bp, Artificial Sequence
  Description of Artificial Sequence Synthetic polynucleotide
gggagagcattgctcgttagtgtcgtctaatcggttttccggtttggtcgttgagctagtgactcggatcatctagg

SEQ ID NO: 31 - DNA, 31 bp, Artificial Sequence
  Description of Artificial Sequence Synthetic polynucleotide
tcgtctcgggatttttcccgtggtcgttcat

SEQ ID NO: 32 - DNA, 77 bp, Artificial Seqeunce
  Description of Artificial Sequence Synthetic polynucleotide
gggagagcattgctcgttagtgtcgtctcgggatttttcccgtggtcgttcatagctagtgactcggatcatctagg

SEQ ID NO: 33 - DNA, 31 bp, Artificial Sequence
  Description of Artificial Sequence Synthetic polynucleotide
tcgtctcgagttgtgattactcgtggtcgta

SEQ ID NO: 34 - DNA, 77 bp, Artificial Sequence
  Description of Artificial Sequence Synthetic polynucleotide
gggagagcattgctcgttagtgtcgtctcgagttgtgattactcgtggtcgtaagctagtgactcggatcatctagg

SEQ ID NO: 35 - DNA, 31 bp, Artificial Sequence
  Description of Artificial Sequence Synthetic polynucleotide
tcgtctcatcgaatggtcggtgtggtcgtta

SEQ ID NO: 36 - DNA, 77 bp, Artificial Sequence
  Description of Artificial Sequence Synthetic polynucleotide
gggagagcattgctcgttagtgtcgtctcatcgaatggtcggtgtggtcgttaagctagtgactcggatcatctagg

SEQ ID NO: 37 - DNA, 22 bp, Artificial Sequence
  Description of Artificial Sequence Synthetic polynucleotide
gggagagcattgctcgttagtg

SEQ ID NO: 38 - DNA, 24 bp, Artificial Sequence
  Description of Artificial Sequence Synthetic polynucleotide
agctagtgactcggatcatctagg

SEQ ID NO: 39 - DNA, 6 bp, Artificial sequence
  Description of Artificial Sequence Synthetic polynucleotide
tcgtct

SEQ ID NO: 40 - DNA, 7 bp, Artificial Sequence
  Description of Artificial Sequence Synthetic polynucleotide
tggtcgt

SEQ ID NO: 41 - RNA, 31 nt, Artificial Sequence
  Description of Artificial Sequence Synthetic polynucleotide
aacgauauggucccggaaguugggccucguu

SEQ ID NO: 42 - RNA, 31 nt, Artificial Sequence
  Description of Artificial Sequence Synthetic polynucleotide
gcaugagugaccauauagguugccgcgcucg

SEQ ID NO: 43 - RNA, 31 nt, Artificial Sequence
  Description of Artificial Sequence Synthetic polynucleotide
gacugcggugcguugaugccguguguugcuc

SEQ ID NO: 44 - RNA, 31 nt, Artificial Sequence
  Description of Artificial Sequence Synthetic polynucleotide
gcguuacagcaguugccacaaggcacguuuu

SEQ ID NO: 45 - RNA, 31 nt, Artificial Sequence
  Description of Artificial Sequence Synthetic polynucleotide
gagccuaugguuguugugugcuccucgcggg